(19) United States
(12) Patent Application Publication
(10) Pub. No.: US 2026/0320384 A1
(43) Pub. Date: Oct. 15, 2026

(54) DEVELOPING A MIXTURE OF HETEROGENEOUS PLANT VARIETIES ESTABLISHED TO MEET A DESIRED TARGET PLANT-BASED INGREDIENT SPECIFICATION

(71) Applicant: BENSON HILL, INC., St. Louis, MO (US)

(72) Inventors: Matthew Brett Begemann, Wildwood, MO (US); Ratna Mukherjea, Ellisville, MO (US); Kyle B. Smith, Lake Saint Louis, MO (US); Jason Bull, Eureka, MO (US); Nicole Choquette, St. Louis, MO (US); Jonah K. Scher-Zagier, Albuquerque, NM (US)

(73) Assignee: Confluence Genetics, LLC, St. Louis, MO (US)

(21) Appl. No.: 19/486,132

(22) PCT Filed: May 22, 2024
(86) PCT No.: PCT/US2024/030613

§ 371 Date: Nov. 20, 2025

Related U.S. Application Data
(60) Provisional application No. 63/503,706, filed on May 22, 2023.

(51) Int. Cl. G06N 3/08; A01C 21/00; G06N 3/042; G16B 20/00; G16B 40/30

(52) U.S. Cl. CPC G06N 3/08; A01C 21/005; G06N 3/042; G16B 20/00; G16B 40/30

(57) ABSTRACT

A method for training a machine-learning (ML) model and subsequently applying the ML model to produce a plant-based product having a functional specification with a first threshold value (first trait) and a second threshold value (second trait). The method uses a processor to collect training data into a database including seed data for a plurality of varieties, including first and second phenotypic traits; to train a ML model using the training data based on a plurality of selected features including the first and second phenotypic traits; to input the first and second threshold values into the trained ML model; and to generate a list of heterogeneous mixture ratios of at least first and second seed varieties ranked based on an aggregate probability that the heterogeneous mixture will achieve the functional specification for the plant-based product after processing of the first and second seed varieties in the heterogeneous mixture ratio.

### RELATED APPLICATION AND INCORPORATION OF REFERENCE

This application is a national phase application and claims benefit of and priority to Patent Cooperation Treaty Application No.: PCT/US24/30613 as filed on May 22, 2024 and U.S. Provisional Patent Application Ser. No. 63/503,706, filed May 22, 2023, the content of both application are hereby incorporated by reference in their entireties.

This application also incorporates by reference patent application PCT/US2024/30517, filed May 22, 2024 and published as WO2024243281 on Nov. 28, 2024; U.S. patent application Ser. No. 19/212,366, filed on May 19, 2024; U.S. Provisional Patent Application Ser. No. 63/650,674, filed May 22, 2024; U.S. patent application Ser. No. 19/211,960, filed May 19, 2025; U.S. Provisional Patent Application Ser. No. 63/651,854, filed May 24, 2024. The contents of the aforementioned applications are hereby incorporated herein by reference in their entireties.

Genomics and selective breeding have been used for decades to develop crops for our food system, but most agricultural companies have focused almost exclusively on increasing the yield of a few crops, resulting in commodity ingredients and a food system based on the quantity of calories available. While focus on quantity is important, that focus has resulted in lower nutrient density and changed flavors. Minimal diversity in ingredient options also led food manufacturers to add costly water- and energy-intensive processing steps, and additives like sugar and salt to make up for attributes that were muted in crops over time.

However, consumers are now demanding food choices with simpler ingredients that benefit their health and the health of our planet. Food- and diet-related health issues, including obesity and diabetes, are some of the most widespread health issues today and continue to increase. More than 65% of American adults are either overweight or obese and, according to the Centers for Disease Control and Prevention, approximately 90% of Americans do not eat the recommended daily amount of fruits and vegetables. Americans spend more on diet-related illnesses than on food itself.

Farmers are similarly demanding improved choices in animal and fish feed to improve the health and management of livestock and fish.

Moreover, the current food system has a substantial environmental impact on the planet. According to an April 2020 report entitled “Agriculture and climate change” prepared by Mckinsey & Company, 27% of total greenhouse gas emissions (e.g., methane and nitrous oxide) are caused by agriculture, with cattle and dairy cows alone contributing eight gigatons of carbon dioxide equivalent (GtCO2e) emissions in 2019. (Accessed Dec. 22, 2021 at https://www.mckinsey.com/~/media/mckinsey/industries/agriculture/our%20insights/reducing %20agriculture%20emissions%20through%20improved%20farming%20practices/agriculture-and-climate-change.pdf.)

At the same time, demand for plant-based solutions to feed the world and improve the environment is growing. Consumers are open to changing their eating habits to minimize further harm to the environment. Moreover, people are actively trying to incorporate more plant-based foods into their diets, especially protein alternatives found in the meat and dairy grocery store sections. NielsenIQ Sep. 9, 2021 article entitled “Growing demand for plant-based proteins” (Accessed Dec. 22, 2021 at https://nielseniq.com/global/en/insights/analysis/2021/examining-shopper-trends-in-plant-based-proteins-accelerating-growth-across-mainstream-channels/).

The largest commercial source of plant protein today is the soybean plant. Other plant-based protein crops include chickpeas, edamame, lentils, peanuts, and peas.

### Soybeans: Generally

Soybeans are believed to have originated on the Asian Continent (glycine soja) where it is believed they were also first domesticated in China (glycine max). Abstract, Hymowitz and Newell, Taxonomy of the Genus Glycine, Domestication and Uses of Soybeans, ECON. BOT. 35, 272-88 (1981). Soybeans are a common field crop with the largest producing countries including the United States, Brazil, Argentina, China, India, Paraguay, and Canada. In the United States in 2020 soybeans were primarily produced in the Western Corn Belt (48.7%), the Eastern Corn Belt (32.7%), and the Midsouth (11.9%) with Illinois and Iowa being the largest-producing states. SETH NAEVE & JILL MILLER-GARVIN, U. MINN., UNITED STATES SOYBEAN QUALITY 2020 ANNUAL REPORT (Published by the University of Minnesota with the support of the United Soybean Board).

Soybean plants produce seed-bearing pods, each generally having two to four seeds. The seeds are harvested and processed either for future planting (i.e., to produce additional soybean plants) or processed into dozens of products (e.g., bean curd, feed for livestock, flour, meal, oil (cooking and industrial)). Soy flours include flour concentrates and isolates, which are the primary protein products of soy.

Soybean seeds are usually planted in rows in soil. According to the 2012 Illinois Soybean Production Guide, soybeans require 55-60° F. soil temperature, an air temperature of at least 68° F., about 25 inches of water, sufficient nitrogen and five months from germination to harvest.

The radical (or root) is the first structure to emerge from a germinating soybean seed. The hypocotyl is the seedling structure that emerges from the soil surface. As the hypocotyl emerges it forms a crook as it pulls the cotyledons (i.e., the plant's first leaves) from the soil. Then, the cotyledons can unfold and begin the process of photosynthesis. Once the cotyledons have emerged from the soil surface the plant is said to be at the VE stage of vegetative development. The VC (cotyledon) development stage occurs once two unifoliate (or single-blade) leaves emerge from opposite sides of the main stem and no longer touch the cotyledons. The V1 (vegetative) development stage occurs once the unifoliate leaves are fully expanded establishing the first node. V2 is defined as the stage wherein a second node (with a trifoliate leaf (i.e., three or four leaflets per leaf)) has formed above the unifoliate node. With the formation of each subsequent node “n” (n=3, 4 . . . ) with fully developed leaves the plant is referred to as being in the Vn development stage. Soybean farmers typically refer to the leaves and stems as the canopy.

The length of time for these vegetative and reproductive stages (discussed below) depends on the plant's maturity group (“MG”) (i.e., the length of time from planting to physical maturity), the soil and air temperatures, and day length. Soybeans are short-day plants (i.e., the soybean plant is triggered to flower as the day length decreases below some critical value, which differs among MGs). See, e.g., LARY C. PURCELL ET AL., U. ARK. DIV. AGRIC. RSCH. & EXTENSION, ARKANSAS SOYBEAN HANDBOOK, ch. 2 (2014). Soybeans planted in Arkansas tend to be MG3 through MG6. Id. In Illinois, where soybeans may be grown in regions traditionally understood to be in MG 2 through MG 5, the 2012 Illinois Soybean Production Guide notes that MG 5 to MG 8 soybeans tend to be determinate (i.e., they cease vegetative growth when the main stem terminates in a cluster of mature pods) and MG 0 to MG 4.9 tend to be indeterminate (i.e., they develop leaves and flowers simultaneously after flowering begins).

Each soybean plant can produce a lot of flowers. The flowers are small and hidden underneath the leaves of the plant. The number of flowers produced depends upon the number of nodes on the main stem and branches with flower-bearing nodes. Not all flowers produce pods. For those flowers that do produce pods whether the resulting pod produces a full complement of seeds requires ample nitrogen, sugar, other nutrients, and favorable environmental conditions.

When a soybean plant begins to flower, it is referred to as being in its reproductive (R) growth stage. Soybeans are a normally self-pollinating crop, in fact, they have a perfect flower structure for self-pollination. Still, bees have been known to be attracted to soybean flowers and cross-pollinated plants. Where cross-pollination is desired breeders need to intervene to prevent self-pollination: the pistil of a soybean plant can become mature and the anthers can begin to shed pollen before the soybean flowers even bloom, breeders seeking to cross-pollinate need to be proactive.

Soybean plants have eight reproductive stages: R1 (beginning flowering/bloom (i.e., at least one flower)), R2 (full flowering/bloom (i.e., an open flower at one of the two uppermost nodes)), R3 (beginning pod (i.e., a pod measuring 3/16 inch at one of the four uppermost nodes)), R4 (full pod (i.e., a pod measuring ¾ inch at one of the four uppermost nodes)), R5 (beginning seed (i.e., a seed measuring ⅛ inch long in the pod at one of the four uppermost nodes)), R6 (full seed (i.e., a pod containing a green seed that fills the pod at one of the four uppermost nodes)), R7 (beginning maturity (i.e., one normal pod has reached mature pod color)), and R8 (full maturity (i.e., at least 95% of pods have reach full mature color)).

As the days get shorter and the temperatures get cooler, the leaves on soybean plants begin to turn yellow, they subsequently turn brown, fall off, and expose the matured pods of soybeans. The soybeans are now ready to be harvested using combines. The header on the front of the combine cuts and collects the soybean plants. The combine separates the soybeans from their pods and stems, and collects them into some container.

After harvesting, the soybeans are processed. The soybeans are cleaned, heat dried, crushed and then flaked. Thereafter, the flake is further processed. The primary method for further processing is referred to as the extraction or solvent process, as it uses organic solvents (e.g., hexane) to recover the soybean oil and protein from the flake. Aside from its substantial use of solvents, this process consumes significant amounts of energy.

### Soybeans: Seed Varieties, Breeding, and Genetic Modification

Today, there are literally thousands of varieties of soybeans. These soybeans are the result of hundreds of years of selective breeding. Selective breeding is the process of selectively propagating plants with more desired traits (often called “phenotypes”) and eliminating plants with less desired phenotypes. Breeding generations are often designed F1, F2, etc. (wherein the “F” stands for “filial”). It may further involve crossing two plants to produce one or more new varieties.

Plant botanists have understood since the days of Gregor Mendel, that plants may exhibit dominant or recessive phenotypes/traits (e.g., seed shape, flower color, seed coat tint, pod shape, unripe pod color, flower location, and plant height). Through his experiments on pea plants, Mendel further taught that the genotype of a particular phenotype is not necessarily correlated because that phenotype may result from homozygous dominant, heterozygous, or homozygous recessive alleles. Where the phenotype is dominant, it will be exhibited by either of the first two zygosities, whereas a recessive phenotype can be exhibited only by the third, homozygous recessive example.

Homozygous genotypes breed true from generation to generation, while heterozygous genotypes do not. Thus, after finding a desirable phenotype, plant breeders work to develop homozygosity in the population, and then release the resulting pure line as a new variety. For example, hybrid varieties are the result of crossing two homozygous, but unrelated, pure lines of a species. The resulting F1 of the cross are all heterozygous. However, by F2 50% of the plants are either homozygous (dominant or recessive) and by F3 heterozygosity is reduced to 25%. Once a desired trait is found in homozygous plants, commercial quantities are produced by replanting the resulting seeds over several generations.

However, it takes time for each generation of plants to grow from seeds to adult plants and time to cross plants once they have produced reproductive organs. These and other practical constraints of biology are natural obstacles for traditional breeding programs and slow the advancement of potential commercial products through the phases of a traditional commercial product pipeline. Moreover, while the foregoing basic principles of plant genetics are relatively straightforward, the issue of creating a commercially desirable variety is complicated by the fact that breeders cannot isolate a particular phenotype from other traits that might be present elsewhere in the plant's genome. Even in Mendel's pea experiments where he worked with only seven traits, each having at least two phenotypes (e.g., green or yellow seed color) existing with the six other traits, each of which also having multiple phenotypes, the number of potential combinations explodes, taking into consideration all the ways the phenotypes of each trait can combine with the phenotypes of all the other traits. In the case of the soybean (i.e., Glycine max), its genome has approximately 1,100M base-pairs packaged into twenty chromosome pairs. Arumuganathan K, Earle ED. Nuclear DNA content of some important plant species. Plant molecular biology reporter. 1991 August; 9 (3): 208-18. Thus, there is an infinite number of potential genetic combinations within the soybean genome. As should be readily apparent, due to the sheer size of a soybean plant's genome, the number of traits/phenotypes, and other practical constraints of biology, traditional plant breeding requires significant time to establish new phenotypes in a population.

Plants may also be genetically modified. Genetic engineering allows for the introduction of a new trait or better control over an existing trait. In 2002, for instance, the majority of the soybean plants grown in the United States were genetically modified for herbicide-tolerance. D. A. Sleper & J. G. Shannon, ROLE OF PUBLIC AND PRIVATE SOYBEAN BREEDING PROGRAMS IN THE DEVELOPMENT OF SOYBEAN VARIETIES USING BIOTECHNOLOGY, AGBIOFORUM, 6 (1&2): 27-32 (2003).

There are two predominant approaches to genetic engineering in plants: the gene gun and the agrobacterium method. Neither of these methods is particularly easy. Under either method, DNA sequencing is generally desirable to confirm that the host cell now contains the new gene and where the gene inserted.

In the gene gun method, the desired gene is coated onto small metal particles and shot within a vacuum chamber using a short, high-velocity pulse of a high-pressure, inert gas (e.g., Helium) toward plants covered by a fine mesh baffle that catches the small metal particles while allowing the gene to continue into the target cell.

In the agrobacterium method, the tumor-inducing region is removed from transfer DNA and replaced with the desired gene and a marker, which are inserted into the tissue of an organism usually by direct inoculation with a culture of transformed Agrobacterium. An antibiotic medium is subsequently introduced to kill the Agrobacterium and remove the marker. Only tissues expressing the marker will survive and possess the gene of interest. These tissues are then grown using tissue culture techniques until a plant is grown and produces seeds.

Among the more desirable characteristics that have been selectively bred in soybeans are increased yield and increased tolerance to various potential environmental stressors (e.g., insects, drought). Unfortunately, according to the United States Soybean Quality 2020 Annual Report (conducted by Naeve and Miller-Garvin of the University of Minnesota), while soybean yields have significantly increased in the United States over the last thirty years, the amount of protein contained in those soybeans has substantially declined over the same time period.

Although genetic breeding programs have yielded some success, there are limits to the efficacy and reach of gene editing programs to create novel lines of plants.

### Limitations of Gene Editing and Selective Breeding to Create Plant-Based Ingredients

Despite the advances in modern gene editing and selective breeding techniques, there are still limitations that hinder the development of novel soy-based ingredients. Genetic editing is expensive, unpredictable, and, even when successful, can take years to self-cross promising lines of soy (or other crops) into a stable, field-ready variety that can be grown on a commercial scale. The development of soy-based ingredients which are tailored to meet consumer needs or particular plant-based ingredient specifications can take years to create, stabilize, and scale. This can limit innovation and investment into new and needed lines of soy and other commercial crops.

Likewise, selective breeding practices have limitations. Certain varieties are difficult to cross-breed and the creation of F1, F2, and subsequent generations is limited by the growing season and the growing space.

For example, there are certain threshold amounts of grain components that are commercially valuable. For example, a soybean variety that can reliably deliver a mean harvest dry weight yield of at least 55 bushels/acre with a grain content profile capable of achieving a white flake protein content of at least about 59.5% and an oil content of at least about 18% is highly desirable. However, single varieties of soybeans cannot typically achieve those thresholds at an industrial scale. This may be due to various reasons, including genetic and physiological antagonism between the features that make conventional crop improvement methods ineffective. For example, there are certain antagonistic traits in soybean varieties (as well as in other plants) that limit the development of novel varieties. For example, as has been long known in the art, selective breeding for an increase in a particular variety's protein content results in a corresponding decrease in that variety's yield and oil content. Conversely, selectively breeding for an increase in a variety's oil content corresponds to a decrease in protein content. Selective breeding for an increased yield corresponds to a decrease in protein content. These traits can be mutually desirable for a breeder or necessary for a particular plant-based ingredient specification, but still remain antagonistic with one another due to limitations of the soybean's biology.

Thus, to create a novel line of soy that has an increased yield, such that growers will deem the variety economically valuable to cultivate, and which has a sufficiently high protein content to meet ingredient specifications (and customer) demands, is challenging, if not impossible to achieve with selective breeding alone. Historically, to supplement selective breeding programs, gene editing techniques have been used to create novel lines that would otherwise take many generations of breeding to achieve. However, despite some developments from selective breeding and gene editing techniques, there are still limitations to the speed at which new lines of soy (and other plants) can be developed. Thus, modern selective breeding and gene-editing techniques are limited and, in many cases, unable to meet the plant-based ingredient specifications required to create soy-based foods and other products (such as animal and fish feed). This is particularly true when developing novel strains with antagonistic traits, such as increases in protein, oil, and yield.

### Challenges of Mixed Varieties of Soybeans in a Shared Field

For hundreds of years, it has been standard to cultivate a single variety of a single crop in a single field. Individual crop varieties require specific growing conditions, including field conditions, spacing, nutrient requirements, water requirements, sun requirements, and other requirements to produce the greatest yield. Thus, although many growers regularly cultivate multiple varieties of soy, each variety is traditionally cultivated in a best-fit field, and varieties are not intermingled. See, e.g., Seth L. Naeve, Choosing the best soybean varieties for your field, U. MINN. EXTENSION, https://extension.umn.edu/soybean-variety-selection/choosing-best-soybean-varieties-your-field (last visited May 22, 2024). Typically, growers select a variety of soy (or other crop variety) that will have desired characteristics and phenotypes that will generate sufficiently high yields to make the plantings commercially profitable.

Some varieties are better adapted to specific field conditions. As new strains of soy (and other crops) have been developed, corresponding agricultural practices tailored to those specific varieties have been fine-tuned to allow farmers the most efficient means to select, plant, and harvest a particular variety of soy (or other crops). Farming equipment, including machinery used to plant, feed/water, harvest, transport, and process soy can be calibrated to increase the yield of a particular crop variety. At the same time, the demand for soy-based products has increased as many consumers are seeking meat alternatives. Soy-based meat alternatives, as well as other popular soy-based products (including animal and fish feeds) require specific characteristics to meet consumer demands. For example, some soy-based products require higher-than-normal protein levels, other products require soy that has a particular oil content, and yet other soy-based products may require soy with a particular water content. Therefore, in order to meet food ingredient specifications, a grower must choose which soy varieties will both meet specific product requirements while balancing the need to select varieties which can be grown successfully in their fields. This limits the varieties of soy that a grower can select and the commercial viability for farmers who seek to grow soy to be used as food ingredients for specific soy products.

Furthermore, differences in maturation times, seed sizes, plant growth rates, plant-spacing requirements, and plant heights make it impracticable to comingle different varieties of soy in a planted field. In order to grow multiple varieties of soy, or any other crop, farmers have to dedicate an entire growing area to a single, homogeneous crop variety. This allows farmers to plant, grow, and harvest a single variety of soy with the required care, growing materials, and harvesting equipment, as well as to streamline storage and transportation processes. The result of this is highly homogenous fields, typically containing a single variety of soy, or other crop. Although it is possible for farmers to plant a field with multiple varieties of a crop, these crops will be segregated into areas or strips of a field or growing area. Further, because each variety of soy has a different profile of oil content, protein content, water content, and expected yield, the types of ingredients and foods a grower can produce is limited to the types of soy which they plant in a given field or growing area. This makes it practically impossible for a grower to cultivate a single field soy to meet a specific product specification.

One potential solution which was never previously practiced would have been to blend existing varieties of soy products to meet a specific plant-based ingredient (e.g., food, feed and the like) specification. This involves using processed soy products from multiple varieties, grown in multiple different locations to create a desired product specification. This is impracticable and inefficient on a commercial scale, however, because farmers would have been required to grow segregated soy varieties in homogenous fields to generate a variety-specific soy product, processed at a variety-specific processing center, and then food scientists would need to combine harvested soy products from multiple growers to fine-tune ratios to meet food ingredient specification requirements. This process would have been inefficient, wasteful, and expensive.

Another potential solution which was never previously practiced would have been to process soy products from multiple varieties at the same time. This option was avoided because processing mixed varieties simultaneously was thought to be problematic for the processing machinery, leading to potential mechanical breakdowns and other potential inefficiencies.

The present invention uses a mixture of multiple varieties throughout the entire lifecycle of cultivation. As used herein, the mixture of multiple varieties of plants may be referred to as a “blend,” “mixture,” or “heterogenous mixture,” but in each case the mixture of multiple varieties will consist of the seeds more than one variety of plant such that the multiple varieties are evenly distributed throughout the mixture. Heterogenous mixtures of plant seeds can be planted together in a heterogenous mixture, grown together as a heterogenous mixture, harvested together by a single harvesting machine, transported as a heterogenous mixture of harvested plants, stored as a heterogenous mixture, and processed together in a single crushing, milling, or other processing facility as a heterogenous mixture, to create a final product that is a heterogenous mixture of soy products. In fact, after various experiments described herein, we now know that the cultivation of a heterogenous mixture selected to meet a specific product specification process can be accomplished without harm to any planting, farming, harvesting, transportation, storage or processing machinery or facilities.

### Using Machine Learning to Improve Agricultural Ingredients

While protein output in soybeans has been decreasing, the demand for plant-based protein has been growing. It has been growing so much that the demand will likely not be fully met using current breeding, genetic engineering, agronomic, and processing technologies. The current commodity food system can take on the order of six to ten years to improve crops with quality attributes, assuming the agricultural industry can even find the genetic synergy to create the right germplasm and then figure out how to best enable the desired breeding.

Machine learning (ML) (and other forms of artificial intelligence (AI)) are already being used to improve certain outcomes in agriculture. One key to successful machine learning is identifying the right types of data to gather and then using that data to train the right type of model. Another key to successful machine learning includes identifying the wrong, unnecessary, or cumbersome data, the inclusion of which is either unhelpful in developing the model or unnecessarily slows down or otherwise makes the training process unnecessarily expensive without sufficient improvement of the model.

ML systems have been used to aid selective breeding and gene editing programs, see, e.g., PCT/US2022/054398; WO2023129746A; PCT/US2022/054267; WO2023129664A3; PCT/US2022/05425; WO2023129653A2; PCT/2022/054403; WO2023129750; US2023/0108027A1; and US2023/0108027A1. However, even with the aid of machine learning, the challenges of gene editing and selective breeding are such that it remains difficult, expensive, and time consuming to meet certain food ingredient specification requirements.

Therefore, there exists a long-felt, unmet need to supplement or replace selective breeding and gene editing programs to be able to predict which varieties of a commercial crop can be developed or combined to meet specific food ingredient requirements.

### SUMMARY OF THE DISCLOSURE

Provided herein is a way to attain industrial harvests of soybeans (and other plants) that achieve a plant-based ingredient with one or more improved traits. In particular, achieving certain trait profiles from a farmer's harvest would be highly desirable and generally has not been attained by current genetics or plant breeding methods. In some aspects, such improved grain composition profiles may be achieved by blending, such as mixing, the grains of at least two compatible varieties so that when the grain is processed, for example, into oil, meal, white flake, protein extract, or protein isolate, the resulting product contains certain thresholds of components valuable to food and/or feed production. While this invention is disclosed in terms of soybeans, it should be understood that any legume-type plant can be utilized instead.

In certain aspects the present specification teaches a method for training a machine-learning model and subsequently applying that model to produce a plant-based product that has a functional specification that meets a first threshold value of a first trait and a second threshold value of a second trait. As should be understood by those skilled in the art having the present specification before them, the methods disclosed herein may be applied in an effort to achieve or exceed threshold values with respect to more than two traits at a time. The method comprises (a) collecting training data into a database, with a processor, wherein such training data comprises seed data for a plurality of seed varieties, such seed data including a first phenotypic trait and a second phenotypic trait; (b) training, with the processor, a first neural network machine learning model using the training data in the database based on a plurality of selected features including the first and second phenotypic traits; (c) inputting, via the processor, the first and second threshold values into the trained first neural network machine learning model; and (d) generating a list of heterogeneous mixture ratios of at least a first seed variety and a second seed variety ranked based on an aggregate probability that the heterogeneous mixture will achieve the functional specification for the plant-based product after processing of the first and second seed varieties in the heterogeneous mixture ratio.

In some aspects, the method may further comprise mixing the first seed variety and the second seed variety into a first heterogeneous mixture based on a first ratio selected from the list of heterogeneous mixture ratios. This method may still further comprise processing the first heterogeneous mixture into the plant-based product.

In other aspects of the invention, the method comprises planting the first heterogeneous mixture of seeds in a first agricultural field, managing plants grown from the first heterogeneous mixture seeds in the first agricultural field, harvesting the first and second seed varieties from the plants in the first agricultural field, and processing the first and second seed varieties harvest from the first agricultural field into the plant-based product.

In some aspects of the present invention, the training data may further include field conditions for a plurality of agricultural fields, such field conditions including a first field condition and the plurality of selected features including the first field condition, the method further comprising designating the first agricultural field for planting the first heterogeneous mixture of seeds whereby the aggregate probability that the first heterogeneous mixture will achieve the functional specification of the plant-based product after processing of the first and second seed varieties from the plants in the first agricultural field takes into account the field conditions of the first agricultural field. As should be understood by those skilled in the art having the present specification before them, the methods disclosed herein may account for more than one field condition at a time.

In some aspects, the training data may further comprise field management techniques, such as planting distances, fertilization, and the like, such field management techniques including a first field management technique and the plurality of selected features including the first field management technique, the method may further comprise providing instructions for use of the first field management technique in association with the first heterogeneous mixture of seeds in the first agricultural field whereby the aggregate probability that the first heterogeneous mixture will achieve the functional specification of the plant-based product after processing of the first and second seed varieties from the plants in the first agricultural field takes into account the first field management technique. As should be understood by those skilled in the art having the present specification before them, the methods disclosed herein may recommend more than one field management technique at a time.

In another aspect of the present disclosure, the methods may further comprise collecting data, by the processor, from the plant-based product after processing of the first and second seed varieties and such training data further comprises produced product specification data. The data collected from the finished plant-based product will be used to train the machine learning system and improve the predictions of the system.

In still another aspect of the present disclosure, the methods may further comprise breeding for a second seed variety by crossing a third seed variety and a fourth seed variety as instructed by the machine learning model. As would be understood by those skilled in the art having the present specification before them, the third and fourth seed varieties may be any varieties of the same plant, in fact they may even be from the same variety (i.e., a self-cross). In particular, the ability to breed to a seed variety that will be but one component in a heterogeneous mixture of plant varieties intended to be combined with a probability of meeting or exceeding the desired specification for an improved plant-based product is believed to decrease the time needed to breed to a commercially stable second seed variety, and thus allow for the production of the desired plant-based product sooner.

In certain aspects, provided is a composition comprising a blend of at least two varieties of a plant (e.g., soybeans). Of course, as should be understood by those skilled in the art having the present specification before them, the composition may comprise a blend of more than two varieties in order to meet or exceed a desired specification for a plant-based product produced from the composition. In some embodiments, the blend refers to a blend of seed varieties for planting. In other embodiments, the blend refers to a blend of grain for harvesting.

In some variations of the foregoing, compatible varieties have (i) relative maturities no greater than 0.75 of each other and/or (ii) compatible herbicide tolerance traits, or both. In one variation, compatible varieties have, or further have, statistically similar amino acid profiles.

In one aspect, provided is a composition comprising a blend of at least two varieties of soybeans, wherein the varieties have (i) relative maturities no greater than 0.75 of each other and (ii) compatible herbicide tolerance traits; and wherein the varieties are blended at a ratio to yield grain that is processed to produce a downstream product having at least one property that is improved.

In certain embodiments, the varieties are blended at a ratio to (a) yield grain that is processed to produce white flakes having a protein content of at least about 59.5%, (b) yield grain having an oil content of at least about 18%, and/or (c) a mean yield of at least about 55 bushels per acre, or any combination of (a)-(c).

In some variations, the blend is a grain blend with improved saponin, higher harvest yield and/or greater protein content. In other variations, the blend is a grain blend with improved flavor, higher yield and/or protein content.

In some variations, the blends provided herein balance yield, protein and oil, and can be used to achieve economic outcomes that are currently hard to achieve using single varieties. Blending as described herein can create economic opportunity beyond genetics of single lines.

In some variations, blending as provided herein is not limited to improving growth or survivability of the plants in the field. Rather, the value chain extends beyond the harvest. The blends herein can achieve a desired combination of performance thresholds in harvested grain that is ultimately delivered to the processor (or ultimately produced by the processor). And while yield of grain from the field may be one of those performance thresholds, blending as provided herein includes optimizing other features that benefit steps after harvest, for example, a quality trait like protein content desired by the processor and potential customers/consumers.

In certain variations, blending occurs substantially at any point in the agricultural process. In one variation, the blends are created during or after harvest. For example, in one variation, the blending occurs by mixing seed before or during planting, e.g., the bag of seed sold to a grower for planting comprises the blend the grower needs to produce a desired grain composition after the seeds are planted and the grain is harvested. In another variation, the blends are created by mixing harvested grain. In certain variations, the blend is created after or during harvest and before the grain delivered to a soybean processor, e.g., between trucks containing harvested grain of different varieties.

In some aspects of the present disclosure, a method of producing a plant-based food ingredient from a blend of plant varieties, wherein the grain has a first trait and a second trait, the first and second traits each having a target value in the plant-based food ingredient is disclosed. As used herein, “plant-based food ingredient” may refer to food for human consumption, as well as to feeds for animal and fish consumption.

The plant varieties may all be soybean, all be some other legume plant, or all be yet another type of plant. Particularly where the plant varieties are soybeans, the first and second traits may be oil and protein content, respectively. The plant-based food ingredient produced may be a flour or other plant meal concentrate. In some variations, the method comprises (a) planting seeds from at least two plant varieties together, (b) growing plants from the seeds to maturity, (c) harvesting grain from the plants without separating the plant varieties, and (d) processing the grain from the plant varieties together to produce the plant-based food ingredient. In some variations of the foregoing, the plant varieties are grown together to maturity over a coextensive area.

In some embodiments, the plant varieties may have relative maturities within a 0.2 to 0.3 range of each other, but in other embodiments, the relative maturities may be as great as 0.75 of each other. In some embodiments, the plant varieties comprise a first variety having (i) the first trait with a value that is higher than the target value of the first trait for the plant-based food ingredient and (ii) a second trait with a value that is lower than the target value of the second trait for the plant-based food ingredient; and a second plant variety having (i) the first trait with a value that is lower than the target value of the first trait for the plant-based food ingredient, and (ii) the second trait with a value that is higher than the target value of the second trait for the plant-based food ingredient. In some embodiments, the plant varieties when planted, grown, and/or processed together yield a plant-based food ingredient with traits that are not achieved from planting any one of the individual plant varieties alone.

The methods herein allow for blending seeds to achieve desired features related to protein content, yield, food functionality and others. For example, in some variations, planting different blends achieves a high yield blend of grain with, for example, a certain combination of protein content and quality protein percentage at the crush facility which will produce a soy protein isolate with certain food functionality features.

In an embodiment, the present disclosure is directed to systems and methods for training a machine learning model and subsequently applying that machine learning model to accelerate speed to market for improved plant-based products utilizing a blend. These potential improvements may comprise increased protein content, decreased oligosaccharides (e.g., raffinose and stachyose), maintaining and/or even improving crop yield, improved consumer experience (e.g., taste, texture, smell), and combinations of the foregoing.

These methods provide significant advantages to agricultural breeding and/or gene editing programs by producing plant-based products (e.g., a processed end product such as flour, meal, white flake, protein extract, or protein isolate) with specifications that cannot be achieved from any one of the individual plant varieties alone.

In another embodiment, to meet a food ingredient specification without having to rely solely on cross-breeding existing varieties with desired traits and/or gene editing techniques, the disclosed system and method provide for the development of a mixture of heterogeneous plant varieties to be co-grown, co-harvested and co-processed. The following steps may be utilized: using machine learning to identify potential varieties of a plant that when mixed are expected to achieve a desired target food ingredient specification, using machine learning to select a particular ratio of more than one variety of a plant, creating a heterogenous mixture of seeds from the more than one varieties of a plant, using machine learning to select preferred growing conditions to cultivate the heterogenous mixture of more than one variety of plants in a common field, growing the heterogenous mixture of a plant in a common field, harvesting the heterogenous mixture of plants together, processing the heterogeneously mixture of plants in a common processing center to create a food ingredient containing food product from the more than one variety of plant, using the food ingredient to meet the target food ingredient specification. Non-limiting examples of desired traits that could be selected from multiple varieties of a plant include: flavor, protein, oil, yield, amino acid, oleic acid, water content, sucrose content (% dry basis), energy content (Kcal/lb), oligosaccharides (% dry basis), hilum color, oil holding capacity (OHC) (g/g solids-micro), water holding capacity (WHC) (g/g solids-micro), least gelation concentration (LGC) (% solids-micro), emulsion capacity (mL/g protein), foaming capacity (FC) (mL/g protein), foaming stability (%), gel texture ((g)-micro scale), gel texture ((g)-large scale), bench Texture (g), moisture (%), protein (% dry basis), crude fat/oil (% dry basis), crude Fiber (% dry basis), ash (% dry basis) and other plant variety traits as will be known in the art.

These and other aspects of the disclosure will be further explained below.

### DRAWINGS

The Detailed Description is described with reference to the accompanying figures. The use of the same reference numbers in different instances in the description and the figures may indicate similar or identical items.

FIG. 1A is a diagram of a system and associated methods for predicting the appropriate composition, field management and processing of heterogenous mixtures of multiple crop varieties to meet specific food ingredient specifications.

FIG. 1B is a diagram of a system and method of training and using a machine learning system to predict desired composition, field management and processing of heterogenous mixes of crop varieties to meet food ingredient specifications.

FIG. 2 is a diagram illustrating the types of data that may be used in association with the machine learning model to facilitate the system and associated methods for predicting the appropriate composition, field management and processing of heterogenous mixtures of multiple crop varieties to meet specific food ingredient specifications.

FIG. 3 is a graphical representation of a type of prediction made by the model to facilitate the present system and methods.

FIG. 4 is a diagram of features that may be used to train one embodiment of the machine learning model for predicting the appropriate composition, field management and processing of heterogenous mixtures of multiple crop varieties to meet specific food ingredient specifications, which may include one or more types of machine learning models depending upon the type of feature data used.

FIG. 5 is a diagram illustrating the process of potential changes to one or more of the machine learning models based on live data collection.

FIG. 6 is a diagram of computer processing components that support the training and subsequent operation of the machine learning models of the present disclosure.

FIG. 7 is a flow chart representing one potential embodiment of the disclosed system and methods.

FIG. 8 is a flow chart representing other embodiments of the disclosed system and methods.

FIG. 9A is a comparison of the statistical distribution of oil and protein traits for two particularly selected varieties of soybeans.

FIG. 9B is a comparison of the seed size distribution for two particularly selected varieties of soybeans.

FIG. 9C is a comparison of the statistical distribution of protein content for stand-alone white flake for two varieties of soybeans.

FIG. 9D is a comparison of the statistical distribution of the protein content for three mixture ratios (50:50; 60:40 and 70:30) of two varieties of soybeans.

FIGS. 10A-C each depict one embodiment of heterogeneous blend.

### DETAILED DESCRIPTION

The present invention now will be described more fully hereinafter with reference to the accompanying drawings, which form a part hereof, and which show, by way of illustration, specific exemplary embodiments by which the invention may be practiced. This invention may, however, be embodied in many different forms and should not be construed as limited to the embodiments set forth herein; rather, these embodiments are provided so that this disclosure will be thorough and complete, and will fully convey the scope of the invention to those skilled in the art. Among other things, the present invention may be embodied as methods or devices. The following detailed description is, therefore, not to be taken in a limiting sense.

In the following detailed description of embodiments of the inventive concepts, numerous specific details are set forth in order to provide a more thorough understanding of the inventive concepts. However, it will be apparent to one of ordinary skill in the art that the inventive concepts within the disclosure may be practiced without these specific details. In other instances, certain well-known features may not be described in detail to avoid unnecessarily complicating the instant disclosure.

As used herein, the terms “comprises,” “comprising,” “includes,” “including,” “has,” “having,” or any other variation thereof, are intended to cover a non-exclusive inclusion. For example, a process, method, article, or apparatus that comprises a list of elements is not necessarily limited to only those elements but may include other elements not expressly listed or inherently present therein.

Unless expressly stated to the contrary, “or” refers to an inclusive or, and not to an exclusive or. For example, a condition A or B is satisfied by anyone of the following: A is true (or present) and B is false (or not present), A is false (or not present) and B is true (or present), and both A and B are true (or present).

The term “and combinations thereof” as used herein refers to all permutations or combinations of the listed items preceding the term. For example, “A, B, C, and combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. A person of ordinary skill in the art will understand that typically there is no limit on the number of items or terms in any combination, unless otherwise apparent from the context.

In addition, use of the “a” or “an” are employed to describe elements and components of the embodiments herein. This is done merely for convenience and to give a general sense of the inventive concepts. This description should be read to include one or at least one and the singular also includes the plural unless it is obvious that it is meant otherwise.

The use of the terms “at least one” and “one or more” will be understood to include one as well as any quantity more than one, including, but not limited to, each of, 2, 3, 4, 5, 10, 15, 20, 30, 40, 50, 100, and all integers and fractions, if applicable, therebetween. The terms “at least one” and “one or more” may extend up to 100 or 1000 or more, depending on the term to which it is attached; in addition, the quantities of 100/1000 are not to be considered limiting, as higher limits may also produce satisfactory results.

Further, as used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

As used herein qualifiers such as “about,” “approximately,” and “substantially” are intended to signify that the item being qualified is not limited to the exact value specified, but includes some slight variations or deviations therefrom, caused by measuring error, manufacturing tolerances, stress exerted on various parts, wear and tear, and combinations thereof, for example.

As used herein, “components” may be analog or digital components that perform one or more functions. The term “component” may include hardware, such as a processor (e.g., microprocessor), a combination of hardware and software, and/or the like. Software may include one or more computer-executable instructions that, when executed by one or more components, cause the component to perform a specified function. It should be understood that any and all algorithms described herein may be stored on one or more non-transitory memory. Exemplary non-transitory memory may include random access memory, read only memory, flash memory, and/or the like. Such non-transitory memory may be electrically based, optically based, and/or the like.

In some aspects, provided herein are ways of achieving harvests with certain desired features, for example, a harvest of high-yield and high-protein grain wherein the harvest comprises more than one variety. In certain embodiments, seeds from two different varieties with complementary traits are planted together so that when they are harvested, the grain is a blend of both varieties, and the blend achieves certain desired thresholds. In other embodiments, the grains from two varieties are harvested separately and then blended together before being crushed. In certain variations, computer modeling, artificial intelligence, etc. may be used to determine which varieties to blend, when they are harvested, and when the grain is blended.

### Heterogenous Mixtures of Seeds

In some embodiments, at least two varieties (e.g., V₁, V₂ . . . . Vₙ), including, for example, at least two compatible varieties (as described above), are blended at a ratio to (a) yield grain that is processed to produce white flakes having a protein content of at least about 59.5%, (b) yield grain having an oil content of at least about 18%, and (c) obtain a mean yield of at least about 55 bushels per acre. For other blends, it is contemplated that the white flake may have a protein content of at least about 45%, an oil content of at least about 10%, and a mean yield of at least about 45 bushels per acre. For other blends, it is contemplated that the white flake may have a protein content of at least about 46.5%, an oil content of at least about 18%, and a mean yield of at least about 55 bushels per acre. For other blends, it is contemplated that the white flake may have a protein content of at least about 44%, an oil content of at least about 22%, and a mean yield of at least about 60 bushels per acre. For other blends, it is contemplated that the white flake may have a protein content of at least about 42%, an oil content of at least about 22%, and a mean yield of at least about 65 bushels per acre. For other blends, it is contemplated that the white flake may have an oil content of at least about 23%, and a mean yield of at least about 65 bushels per acre. For other blends, it is contemplated that the white flake may have a protein content of at least about 41%, an oil content of at least about 21%, and a mean yield of at least about 65 bushels per acre. Blends are also contemplated wherein the producer does not care about the resulting oil content in favor of high protein and good yield.

It should be understood that white flakes are generally made after dehulling and pressing soybeans out into thin flakes. This ruptures cells and releases the oil within, which can then be extracted from the flake to create an oil fraction and a defatted (white) flake portion. The resulting white flakes may have a broad range of applications in downstream processes.

In other embodiments, at least two varieties (including, for example, at least two compatible varieties as described above) are blended at a ratio to yield grain that is processed to produce a plant-based product having at least one property that is improved as compared to the weighted average of the at least one property of a processed form of each compatible variety alone. As used herein, the term “improved” may refer to a plant-based product that (a) meets a specification which previous plant-based products did not, (b) has an increased presence of a particular trait, (c) is more appealing to consumers, (d) has more of a particularly desirable functionality as compared to previous products, (e) has less of a particularly undesirable functionality as compared to previous products, (f) is more affordable to produce as compared to previous products, and/or (g) represents any other improvement to a food ingredient product, as would be understood by those skilled in art having the present specification before them, as well as any combinations of the foregoing. In some variations, properties of the plant-based products may include water holding capacity (WHC), oil holding capacity (OHC), least gelation concentration (LGC), emulsion capacity, foaming capacity (FC), foaming stability, micro texture, bench texture, gel texture, protein dispersibility, nitrogen/protein solubility, or viscosity, hilum color, or any combination thereof.

In some embodiments, the ratio of two varieties is at least 5:95, at least 10:90, at least 20:30, at least 30:70, or at least 40:60, at least 50:50, at least 60:40 or least 70:30; or between 50:50 and 90:10, between 50:50 and 80:20, or between 60:40 and 70:30, such that the blend yields grain that is processed to produce a downstream product having at least one property (such as the aforementioned properties) that is improved.

In some variations, such downstream products may include a food or feed ingredient or product, a protein isolate, other protein product, soybean seed hulls, meal, flake, flour, fiber, sugar, or oil product, or a food, feed, aqua, or industrial product, or any combination thereof.

In certain embodiments, two varieties (including, for example, two compatible varieties as described above) are blended together. In some variations, one of the two compatible varieties is a higher protein variety and the other is a lower protein variety. In certain variations, one of the two compatible varieties is an ultra-high protein variety (Variety 1) and the other is a high protein variety (Variety 2). In one variation of the foregoing, the ratio of Variety 1 to Variety 2 is at least 50:50, at least 60:40 or least 70:30; or between 50:50 and 90:10, between 50:50 and 80:20, or between 60:40 and 70:30.

### Timing

In some embodiments, the blending is done at or before planting, such as while the varieties are seeds, for example a heterogeneous blend in a bag that the grower empties into a planter. These heterogeneous blends are planted as seed, grown as plants, and harvested as grain all as a heterogeneous blend. In some embodiments, the varieties are blended as they are harvested from the field. This can be accomplished, for instance, by planting each variety in separate sections, e.g. strips, but close enough together that they can be harvested simultaneously by the combine head preferably in a predictable approximate ratio. The combine harvester head will span from one variety to the other, and thus, in these embodiments, blending occurs simultaneously with the cutting, threshing, and auguring and conveying that occurs in the combine as the varieties are harvested together.

In other embodiments, blending occurs after the varieties have been grown and harvested separately at some point before being processed in the processing facility. For example, In certain of these embodiments, the varieties may be grown in different sections in the same field and can be combined in a bin or when loaded onto a truck for transportation. In certain of these embodiments, the varieties may be grown in different fields entirely and at least one of the varieties is first transported to another location before blending occurs. Blending can occur at substantially any time or place before the blend is processed.

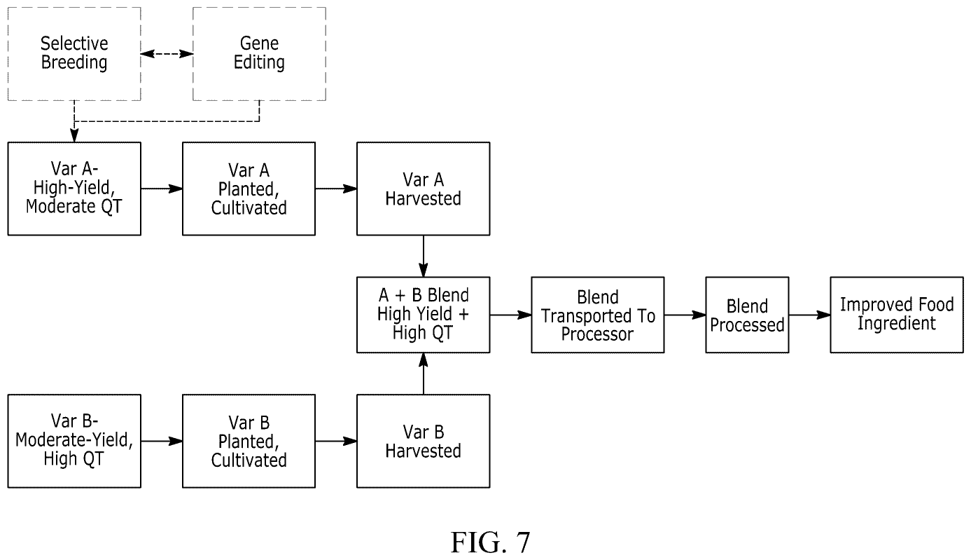

As illustrated in FIG. 7, the benefit from blending is not exhausted at harvest. For example, yield benefit may be realized at harvest and/or at processing, but quality trait benefit can be realized later, e.g., during/after processing. The quality trait benefit may be any trait that the user desires.

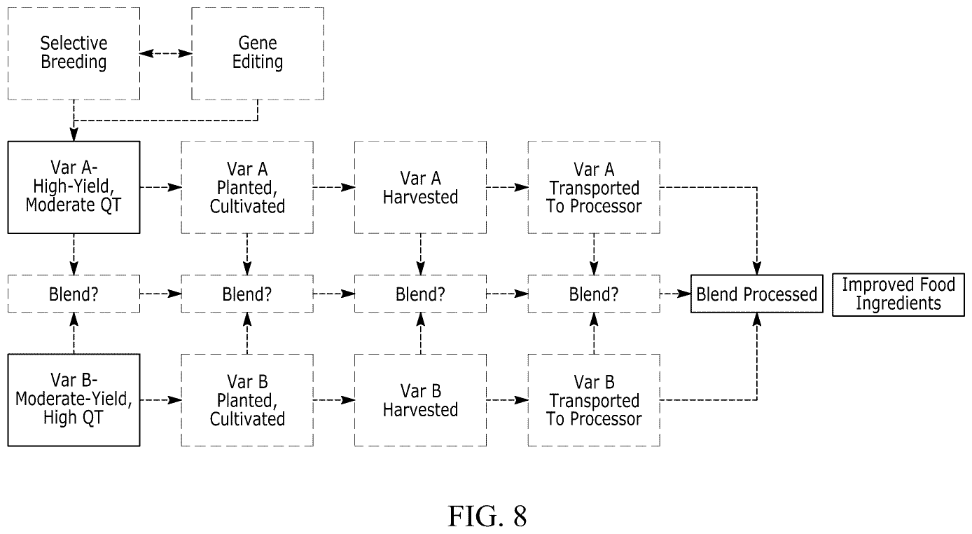

As illustrated by FIG. 8, the blending may occur at any time during the agricultural process. Thus, nodes and internodes in this figure are dashed to indicate they become points where the system can be adjusted. Due to this flexibility, the invention herein can be used in conjunction with computer/artificial intelligence (AI)/prediction systems to adjust (e.g., in real time) which varieties are blended and when in the process the blend occurs. Each dashed line above indicates a point where, for example, an AI system can make a prediction and recommendation for how to proceed. For example, based on a set of product specification thresholds, a computer/artificial intelligence (AI)/prediction system could predict which two varieties should be blended together at planting to produce the desired result, including the optimized ratio of the blend.

Furthermore, if something happens during the growing season to throw that prediction off (like a drought or flood, or a change in the market that dramatically changes the desired product specification), the computer/artificial intelligence (AI)/prediction system can use data collected during the growing season to predict what the harvested blend will be as the plants grow and then recommend an additional blending step after harvest that should be made. This additional blend could involve adding more of a first variety or a second variety, or even a third variety, a fourth variety, etc., whether it is grown at the same location as the blend or grown elsewhere and combined before processing. Thus, the process of blending described herein provides a new way to plant, grow, harvest, and process quality grain.

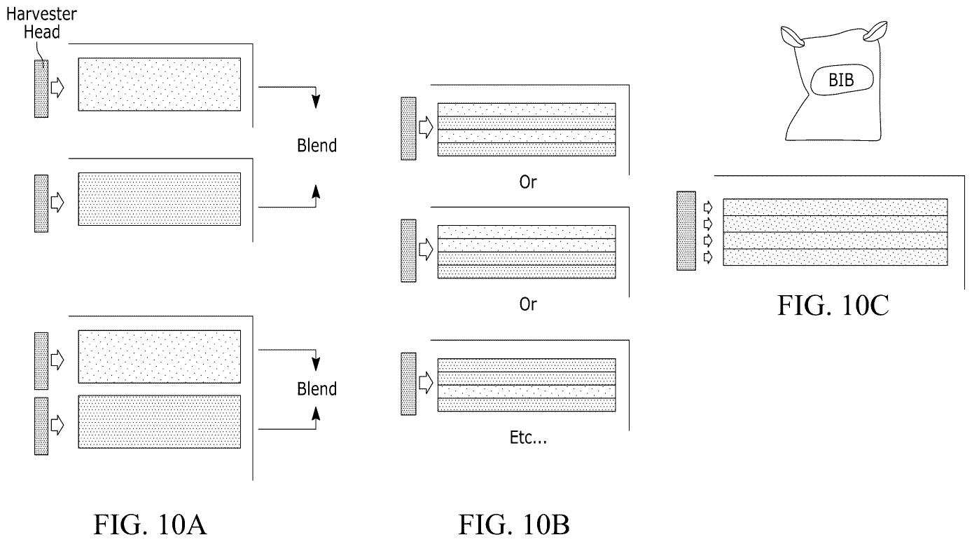

With reference to FIG. 10A, blending occurs after harvest, whether the two varieties are grown in separate fields (top) or separately but in the same field (bottom). In either case, the combine harvests all of one variety, then the other variety, and the two varieties are kept separate until they are blended after harvest.

With reference to FIG. 10B, two varieties are planted in sections in the same field, one variety per section (in this case, strips). The harvester harvests at least some portion of both varieties simultaneously. In certain embodiments, the two varieties are plated in alternating strips and the harvester collects multiple strips of each variety at the same time (top). In certain embodiments the varieties are planted in sections, one variety per section, but the sections of both varieties are harvested simultaneously. In certain embodiments, the varieties are not planted in equal proportions.

With reference to FIG. 10C, the blend occurs in the bag (a “blend in bag” or BIB) and the varieties are intermingled together as they are grown. In such embodiment, the varieties are not separated by strips or sections.

When the varieties are grown together in the same location (e.g., as in FIGS. 10B and 10C), the architecture of the varieties and competition between them produces a harvest composition that was previously unpredictable. For example, normally varieties are harvested as in the top diagram of FIG. 10A (or FIG. 10B scheme that closely approximates separate growing locations).

To determine what ratio to blend the two varieties, various ratios of blends may be tested to arrive at an optimal ratio. However, working backwards from that ideal finished ratio to predict how to achieve it using the approach in FIG. 10B or FIG. 10C can be difficult and complicated, as there is unlikely to be a 1:1 correlation between the ratio of the two varieties that need to be planted in the field to achieve the ratio desired after harvest. In some variations, synergy may be achieved via a field growth interaction of the plants when grown/harvested together and a post-processing/food functionality interaction that occurs when the two varieties are processed together.

As FIG. 8 shows, blending of multiple varieties of a single plant may occur throughout the cultivation lifecycle, including before planting, such that a heterogenous mixture is planted in a single field, during harvest, such that a heterogenous mixture of plants is harvested together or by a single harvesting machine, during transportation following harvest, such that a heterogenous mixture of harvested beans or grain are comingled in a single transportation vessel, during processing, such that a heterogenous mixture of harvested beans or grain are processed together in a single processing facility. Creating a heterogenous mixture of multiple varieties of a plant may occur any stage of cultivation as may be necessary, or as may be recommended by the disclosed machine learning system 150. Blending may occur multiple times and of multiple varieties throughout the cultivation lifecycle, or at a single instance (such as prior to planting or during processing) as may be desired or recommended by the disclosed machine learning system 150.

### Machine Learning, Generally

The term “machine learning” (a type of artificial intelligence (“AI”)) generally refers to computer algorithms that may learn from selected features of pre-existing data and then make predictions about new data. Thus, machine-learning tools operate by building a model from example training data, which, for example, can be used to model an environment based on that training data and then make decisions or predictions without explicit instructions. Different machine-learning tools may be used. Deep learning or deep structured learning is a type of machine learning that can use artificial neural networks (e.g., inspired by biological systems) with representation learning. Representation learning is a set of techniques that allows a system to automatically discover representations needed to detect features in future sets of data. The learning of features is generally thought to be either supervised or unsupervised, although a hybrid of these approaches is also possible.

In “supervised learning,” a “teacher” presents the computer with the desired outputs given a set of example inputs. This is generally thought to involve classification and regression, which can be accomplished using one or more approaches including, but not limited to, decision trees, ensembles (e.g., Random Forest), nearest neighbors algorithm, linear regression (including, for example, average and weighted average), gBLUP (genomic best linear unbiased prediction), lasso (least absolute shrinkage and selection operator), lasso LARS, Ridge regression, Elastic Net, Naive Bayes, Artificial neural networks (ANN or NN), logistic regression, perceptron, Relevance vector machine (RVM), and Support vector machine (SVM). Generally, the approach to supervised learning used depends on the data set, among other issues involved in this choice is the amount training data available, the dimensionality and heterogeneity of that data, redundancy in that data, the interrelations between data elements, and the amount of noise present in the output.

In “unsupervised learning,” the computer is left to find any naturally occurring patterns within the training data. This can be accomplished by using one or more approaches including, but not limited to, clustering (i.e., automatically grouping the training examples into categories with similar features), anomaly detection, principal component analysis (i.e., automatically identifying features that are most useful for discriminating between different training examples and then discarding the rest), self-organizing feature maps, and latent variable models. Clustering methods include hierarchical clustering, k-means, mixture models (i.e., a probabilistic model that represents the presence of subpopulations within an overall population), DBSCAN (density-based spatial clustering of applications with noise), expectation-maximization, BIRCH, and CURE.

One or more of the foregoing supervised and unsupervised machine learning approaches may be used by the present system and methods in parallel or seriatim using the same training data or subsets thereof. Where subsets are used the scope of any such subset may be selected for use with the particularly selected training data within that subset with reference to the pluses and minuses of one or more of the particular approaches to machine learning. Where multiple machine learning approaches are used in parallel (i.e., stacked) a decision-making model is preferably introduced to mediate between the probability assessments provided by the multiple machine learning models toward providing a single list of recommended actions (e.g., desirable plant crosses, gene editing targets, crop management techniques).

### Heterogenous Mixture of Varieties Aided by Machine Learning.

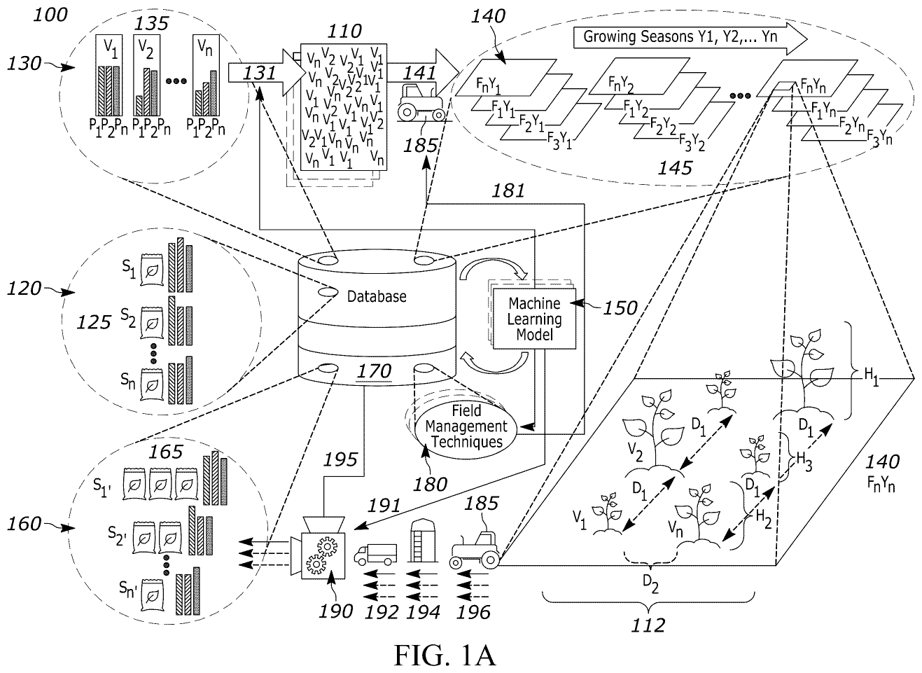

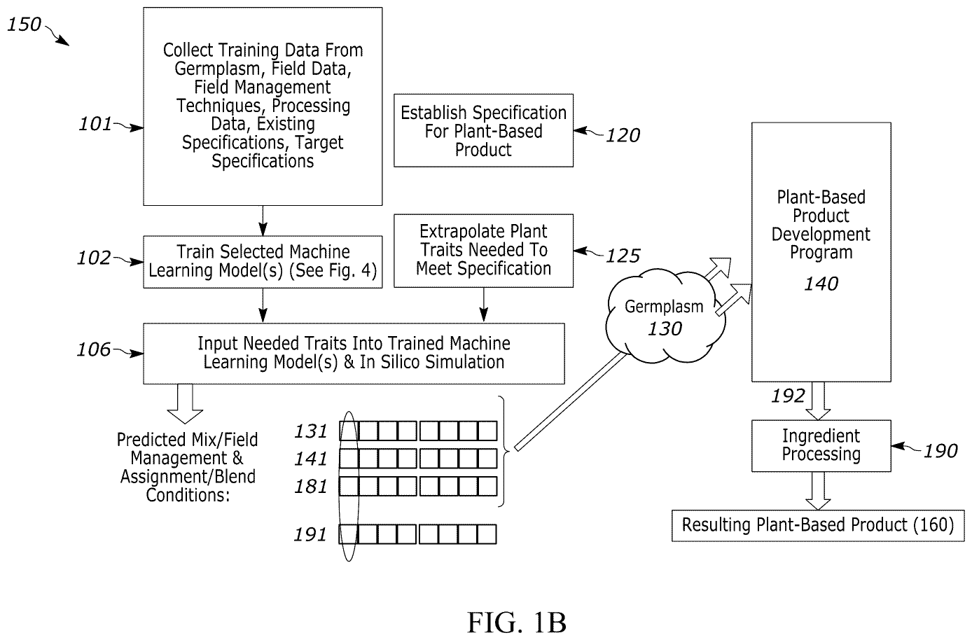

The system and method 100 disclosed in FIG. 1A will be desirable in some instances where it may be difficult or impossible to achieve or exceed a desired product specification using existing plant varieties and breeding and/or gene editing to achieve that result is not readily accessible (because, for instance, it will take too long to achieve a stable, field-ready variety that meets the desired product specifications that can be grown on a commercial scale). For example the following non-limiting factors make it difficult to cross-breed plant varieties: whether the variety is herbicide-traited or non-herbicide traited, whether the reproduction of the variety is determinant or indeterminant, whether the variety is a dwarf or non-dwarf type, the relative maturity (i.e. <0.05), disease traits and susceptibility, seed size, flower color, pubescence color, pod wall color, hilum color, and those other traits as would be obvious to one of ordinary skill in the art. Effective cross-breeding for plant varieties with incompatible characteristics, such as those listed herein, may take years or be all-together impossible. Even if two varieties of a commercial crop each have desirable traits that theoretically could be crossbred, these limitations make it unlikely that crossbreeding could be successful.

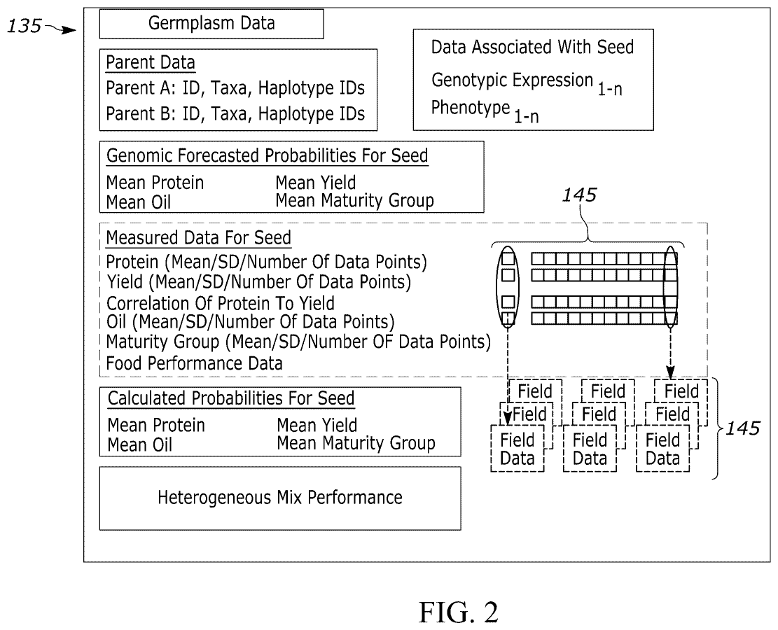

The database 170 stores collected data from a variety of sources. As shown in FIGS. 1A and 2, the data stored may include, but is not limited to, data related to seed varieties 135, field conditions 145, target specification 125, produced product specifications 165, field management techniques 185, and grain processing data 195. As illustrated in FIG. 2, seed variety data 135 may include information from a variety's genome, phenotypic traits such as protein content, yield, oil content, oligosaccharide content, saponins content, amino acid profile, functional properties, dwarfism, determinant/indeterminant, seed size, hilum color, flower color, pubescence color, pod wall color, and longitudinal data for any of the foregoing. These data are used to iteratively and continuously train the disclosed machine learning model 150 such that the data are regularly collected and stored into the database 170, and such that those data are regularly used to train and train the machine learning model 150 to improve performance and prediction abilities of the machine learning model 150.

FIG. 2 further illustrates that field data 145 associated with a particular seed variety may also be stored in association with the seed variety data. In other words, for each field in which has a seed variety has been grown, the seed and field data collected from that seed growing in that field may be associated with the seed variety and, as illustrated such data may be longitudinal.

As depicted in FIG. 1A, the disclosed machine learning model 150 can aid in the selection 131 of varieties 130 of a particular crop to create a heterogenous mixture 110 of seeds to be planted 141 in a particular field 140, and grown, harvested 196, stored 194, shipped 192, and processed 191 via the processor 190 together to meet or exceed a desired food ingredient specification 120 (e.g., S₁, S₂, . . . . Sₙ).

Multiple varieties of a plant 130 may be analyzed by the disclosed machine learning system 150. Varieties 130 may include existing, known lines of a particular plant, or they may be virtual or anticipated varieties that are expected based on machine learning predictions or other genetic breeding lines. Variety data 135 may be stored in a database 170. As shown in FIG. 1B, these variety data 135 (i.e., germplasm data) can be part of the data 101 used to further train 102 the machine learning model 150.

As shown in FIG. 1A, each variety 130 of a plant will have specific phenotypic traits (P₁, P₂, . . . . Pₙ), these traits may be antagonistic to one another, such as, protein content and yield, making the particular varieties 130 ill-suited for cross-breeding programs. The machine learning model 150 is trained based on the variety data 135 (and other data stored in database 170, as discussed herein) to select 131 a mixture 110 of multiple seed varieties 130 to plant 141 in a given field 140. In this manner, the machine learning model 150 predicts a ratio of more than one variety 130 of plants that are projected by the machine learning model to result in a target food ingredient specification 120 when ultimately processed together into a resulting plant-based ingredient.

As shown in FIG. 1A, a heterogenous mixture 110 of seeds (V₁, V₂, . . . . Vₙ) may involve any number of varieties 130 of seeds of a given plant. The ratio of multiple varieties predicted by the machine learning model 150 may be any ratio of seeds for more than one variety of plant as projected by the model to meet (or even exceed) a desired target food ingredient specification 120. For example, it may be that two varieties 130 of a plant can be combined in a heterogenous mixture 110 of seeds to meet a specific target food ingredient specification 120. Alternatively, the machine learning model 150 may predict that more than two varieties 130 of plant seeds may be combined in a heterogenous mixture 110 to meet (or exceed) a specific target food ingredient specification 120.

The machine learning model 150 may also predict (141), based on data from the database 170, which field 140 the heterogenous mixture 110 of seeds should be planted in to meet or exceed the desired food ingredient specification 120. As is known in the art, each growing field 140 has certain conditions related to soil type, temperature, geographic location, aridity, and other conditions as is known in the art, which may impact which varieties of a certain plant are best suited to grow in that particular field 140. The machine learning model 150 may predict which field 140 a particular heterogenous mixture 110 of seeds should be planted 141 in order to have the highest likelihood that a given food ingredient specification 120 can be met. Field data 145 can be part of the data 101 used by the machine learning model 150 to further train 102 the model 150. Field data 145 may include data from specific fields 140, data related to growing seasons (Y₁, Y₂, . . . Yₙ), as well as combined data from specific field for a given growing season (F₁ Y₁, F₁ Y₂, . . . . Fₙ, Yₙ). These data are all collected and stored in the database 170 and used to further train 102 the machine learning model 150 improve future predictions to meet (or excess) target food ingredient specifications 120.

As would also be understood by those skilled in the art having the present specification before them, each plant variety may have previously been managed differently when grown independently in an agricultural field. For example, Variety A may have been observed to grow better when the seeds of this variety are planted at a distance of D_{Va} whereas Variety B may have been observed to grow better when the seeds of this variety are planted at a distance of D_{Vb}, which is different from D_{Va}. Similarly, Variety A and Variety B may grow to different average heights. Furthermore, Variety A and Variety B may have complementary or even competing agronomic behaviors. For example, some varieties of soybeans are known to fix nitrogen better than other varieties of soybeans; some have broader leaves than others, and some grow taller than others. The model 150 may utilize the opportunities that these complementary and/or competing agronomic behaviors offer in determining combinations, ratios, fields, and field management techniques.

After planting 141, the heterogenous mixture 110 of seeds will mature into a heterogenous mixture of plants 112. Field management techniques 181 will be used by growers to ensure the heterogenous mixture of seeds/plants/grain 110/112/192 successfully grows to maturity. Data related to field management techniques 185 is stored the database 170, and as shown in FIG. 1B can be part of the data 101 iteratively used to continuously train 102 the machine learning model 150 as new field management techniques 181 are fined-tuned. In turn, these field management data 180 can be used to train the machine learning model 150 such that the model 150 can make improved predictions about which heterogenous mixtures 110 of seeds can be planted using field management techniques 181 to meet a given target food ingredient specification 120 for future heterogenous mixtures 110.

As shown in FIG. 1A, when the mature heterogenous mixture of plants is ready for harvest, a grower can use a single tractor or combine harvester 185, or other known means of harvesting, to harvest 196 all of the mature heterogenous mixture of plants 112 together. As is known in the art, some varieties of plants may grow taller or be bushier than other varieties. This will impact the ratio of the total mass of plants harvested, with taller, bushier plants representing a greater percentage of the total plants than the correlated percentage of planted seeds. As shown in FIG. 1B, field data 145 related to plant size and bushiness is stored in the database 170 and can be part of the data 101 used to train 102 the machine learning model 150 such that the machine learning model 150 can take into account these differences when making predictions about which ratios of seeds of particular plant varieties should be used to meet a given food ingredient specification 120.

After harvest 196, the heterogenous mixture of harvested heterogenous mixture of plants 112 can be stored 194 and transported 192 together prior to processing 190. The harvested heterogenous mixture of plants 112 can be processed in a single processing center 190 in order to be processed as a heterogenous mixture to create food ingredients. As illustrated by FIGS. 1A and 1B, processing data 195 from the processing 190 are stored in a database 170 and can be part of the data 101 used to iteratively continuously train 102 the machine learning system 150. The machine learning system 150 uses these processing data 195 to make improved processing predictions 191 about which varieties of a plant can be processed together to meet a target food ingredient specification 120 for heterogenous mixtures 110.

The food ingredients that result from the processing center 190, as shown in FIG. 1B, can be analyzed by the machine learning system 150 to determine whether the food ingredients have met the target food ingredient specifications 120. Multiple food ingredient specifications (S′₁, S′₂, . . . . S′ₙ) can be analyzed from a single resulting food ingredient product. It may be the case a particular batch of food ingredient products meets one food ingredient specification 125 (for example S₁) but not another food ingredient specification 125 (for example, S₂). Existing product specification data 165 from the resulting food ingredients data 160 are stored in a database 170 and can be part of the data 101 used to iteratively, continuously train 102 the machine learning model 150.

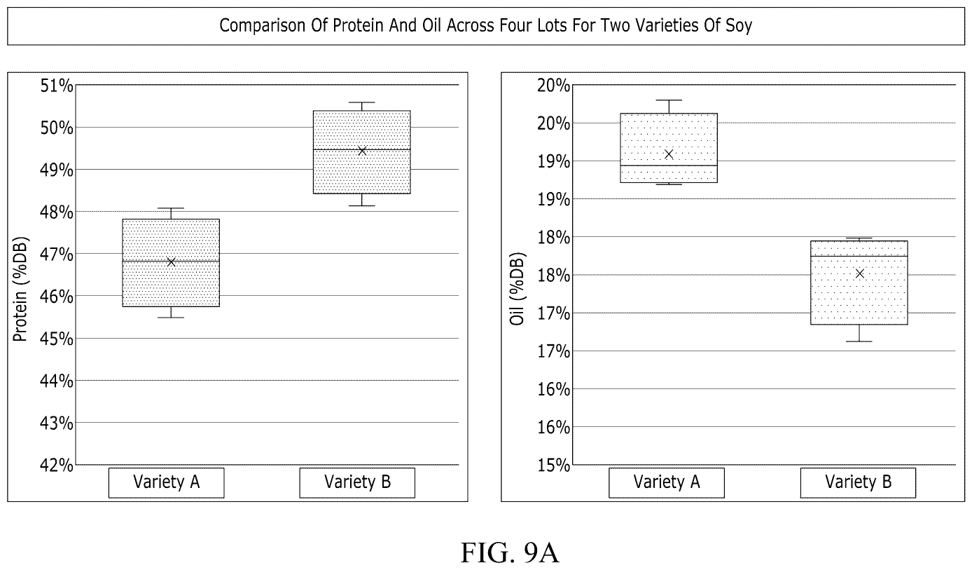

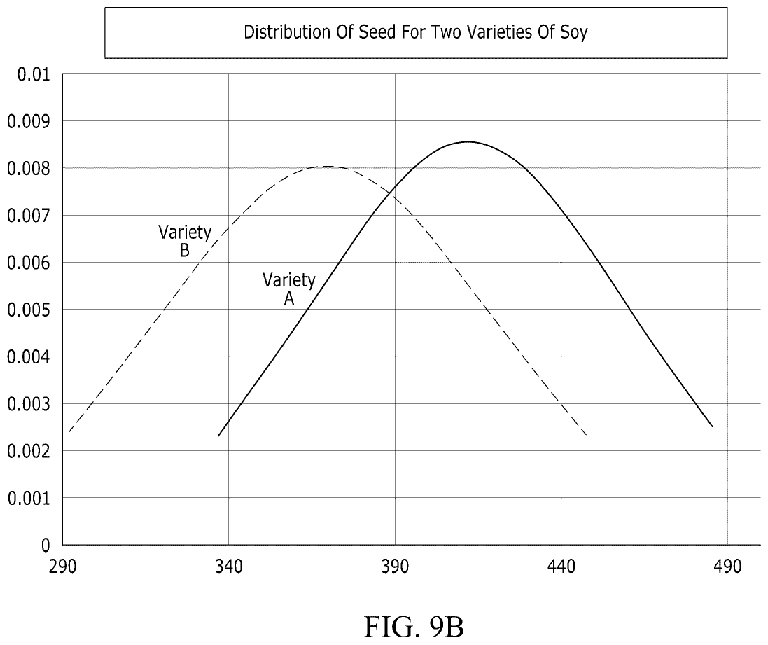

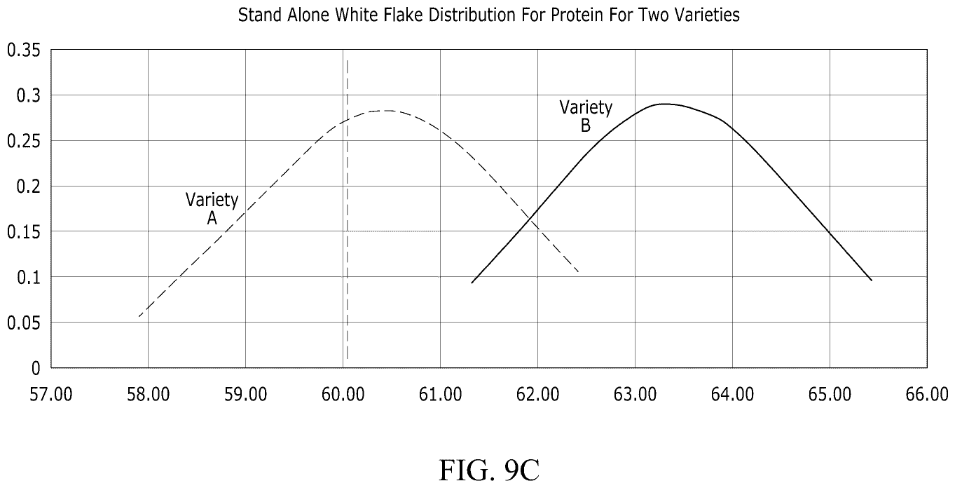

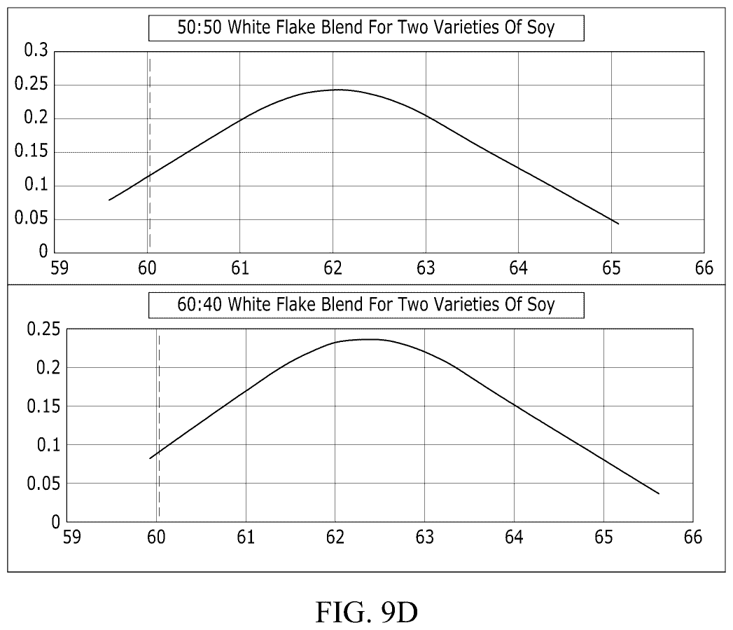

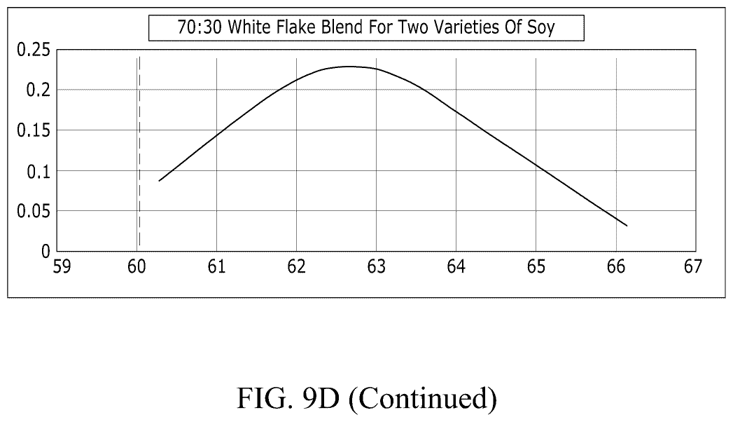

In many cases, some aspects of desired specifications will be easier to achieve through a heterogenous mixture 110 of seeds from multiple varieties 130 than through breeding or gene editing techniques. In other cases, gene editing techniques or selective breeding programs will represent the fastest and/or most efficient way to meet a given plant-based ingredient specification requirement 120. In other cases, gene targets believed to result in the desired traits may be yet unknown, too difficult to edit/modify successfully, provide insufficient improvement of the desired trait, or may otherwise prove undesirable. In some cases, as depicted in FIGS. 8 and 9, a combination of breeding crosses and genetic editing and heterogenous blending will provide the most efficient path to the desired end product specification. In still other cases, breeding, genetic editing, planting location, a heterogenous mixture 110 of seeds, and field management techniques 180 will provide the most efficient (fastest, most cost-effective, or both) path with the highest probability of producing an end product that meets (or exceeds) the target specification 120. In some cases, some or all of the foregoing may be used to achieve a desired specification.

### Training the Machine Learning Models to Meet Target Food Ingredient Specifications

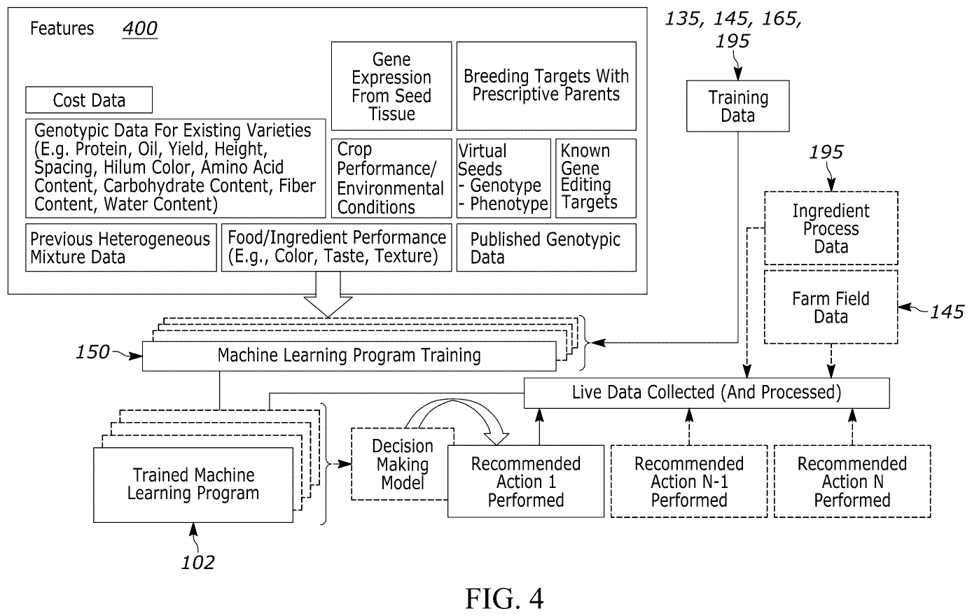

As illustrated by FIG. 1B (as discussed above) and FIG. 4, training machine learning models requires the selection of features 400 and collection of data associated with relevant features in order to appropriately train 102 the machine learning model 150. More particularly, FIGS. 2 and 4 identify various categories and types of data that the inventors believe may play a substantive role in training useful models.

As disclosed in FIG. 1B, the data from the database 170 are used to train 102 the machine learning model 150. Training data may also be collected 101 from one or more of the following: the seed variety (i.e., the germplasm 130) data (e.g., phenotypic, genotypic), breeding program data/targets (e.g., phenotypic, genomic), gene editing program data/targets, as well as publicly available literature and information regarding the plant species underlying the resulting product specifications (165).

As further illustrated by FIG. 1B, a target or desired specification (120) is established for the improved plant-based product and the plant traits needed to meet the specification (e.g., protein content, decreased/muted chemical expression) are extracted from the specification (125). The extracted specifications are input into the trained machine learning model(s) 150 and in silico simulation(s) 106. As a result, lists of desirable predicted 131 heterogenous mixtures having been ranked by probabilities determined by the machine learning model(s) 150 may be produced in order to determine which varieties 130 to plant in a specific field 140. In alternative embodiments, the predicted desirable heterogenous mixtures of multiple varieties may also include recommendations of predicted heterogenous mixtures, selective breeding, and gene editing recommendations used individually or in any combination to meet a product specification. The machine learning model 150 may also predict desirable field management techniques 181 to cultivate a particular heterogenous mixture 110 as a well as desirable planting and growing conditions 141 for a specific field 140. The desirable predicted 131 heterogenous mixtures, the field management techniques 181, and the desirable planting and growing conditions 141 are used to determine the correct heterogeneous mixture of varieties 130 to plant in a particular field 140. Once grown, that heterogeneous mixture of varieties 130 are harvested and moved 192 to ingredient processing 190.

The machine learning model 150 may also predict desirable processing conditions 191 in order to determine which varieties of a plant can be processed together in ingredient processing 190 to meet a target food ingredient specification 120. The results produced by in silico simulation (106) may also be based on the output of one or more component of the physical testing, food testing, historical genetic or phenotypic data of other seeds in the germplasm 135, field conditions 145, specification data 125, 165, field management techniques data 185, and product processing data 195. This historical data may, itself, be real physical testing observations (which may have been obtained from a heterogenous mixture or other in-field growth), calculated from real physical observations, predicted data, the result of in silico simulation, or a combination of one, some or all of the foregoing. The model may adjust based on the source of the data (e.g., real physical observation data versus simulated data versus predicted data). These data are all stored in the database 170 and used to iteratively train the machine learning model 150 and then used in determination/simulation (106).

As further illustrated in FIG. 2, the potentially useful seed variety data may be saved to a seed variety object (or seed vector) 135 that describes the underlying characteristics each unique seed variety in germplasm 130. As particularly illustrated in FIG. 2, the seed variety object 135 is preferably identified by one or more of its germplasm ID, its parentage, genotypic, phenotypic, or other variety-specific data. Seed variety object 135 may be virtual in the sense that it may contain nothing more than the germplasm ID, parentage and variety-specific data. A “virtual” seed variety object 135 may also include genomic forecasted probabilities for the seed such as protein content, yield, oil content, and maturity group, all of which may be represented as their mean values and may have an associated standard deviation.

The data saved to seed variety object 135 may also include measured data for a seed (really a population of seeds sharing a common pedigree). As illustrated in FIG. 2, in the context of soybeans, this measured seed variety object data 135 may include protein, yield, oil, Maturity Group, and food testing protocol data for each instance that seed is grown, i.e., over growing sessions 145 as illustrated in FIGS. 1A and 2. The protein and oil data may be further measured and recorded as to type of protein/oil.

Field data 145 for each instance the seed has been grown and observed may also preferably be associated with this collected data. The field condition data 145 may include information correlating between a plant variety's success in a given field, the related growing conditions. For instance, the field data 145 may include location (e.g., latitude/longitude), soil health (e.g., its microbiome and Nitrogen content), climate data (e.g., rain frequency and amount, sunlight length and intensity, air temperature, soil temperature), and applied crop management practices (e.g., planting depth, plant spacing, planting date, irrigation, fertilizer use). Field data 145 may also include data related to field management growing conditions, such as, for example, mature plant height (H₁, H₂, . . . . Hₙ), planting distance (D₁, D₂, . . . . Dₙ). Where appropriate, the foregoing field (and other) data may be collected and saved longitudinally.

As noted above, the field data 145 collected with respect to any particular growing event may not produce instructive data with respect to all of these variables (e.g., the location of an indoor growing event) or even where all of the variables could have been collected, the data may not have been recorded, entered into the data set, or removed from the data set for various reasons. In this regard, the models selected for use with the overall data set contemplate the potential absence of data points from the overall data set.

These seed object variety data 135, may further include the following, non-limiting information: parental genetic data, include taxa, haplotypes, and other relevant parental genetic information, genomic forecasts for seed traits, such as mean protein content, mean oil content, mean yield content, and mean maturity group. The seed data 135 may further contain information related to cost to cultivate a particular variety. The seed data 135 may also contain information related to live observations for a particular seed variety 130, such as data related to that variety's performance data, and any other data collected relevant to cultivation of that particular variety.

The collection and storage of these data in the appropriate seed variety object 135 in the database 170 is meant to provide as much information about the seed object variety 135 for use in training and subsequently using the machine learning model 150 to make predictions about which seed object varieties 130 may preferably be blended into a heterogenous mixture 110 to meet (or exceed) a specific product specification 120. Thus, the seed variety data 135 may also relate to the features 400 shown in FIG. 4 used to train 102 the machine learning model 150. Non-limiting features 400 that may be used to train the machine learning model (in element 102 of FIG. 1B) are depicted in FIG. 4, and may include: genotypic and phenotypic data, cost data, breeding target data, food ingredient performance and functionality data, heterogenous mixture performance data, crop performance data, field data, and virtual and observed data from breeding programs. One or more of these features 400 may be used to train 102 machine learning models and may be supplemented and/or logically manipulated depending on the target specification requirements 120 or other agronomic goals, as will be understood by skilled in the art having the present specification before them.

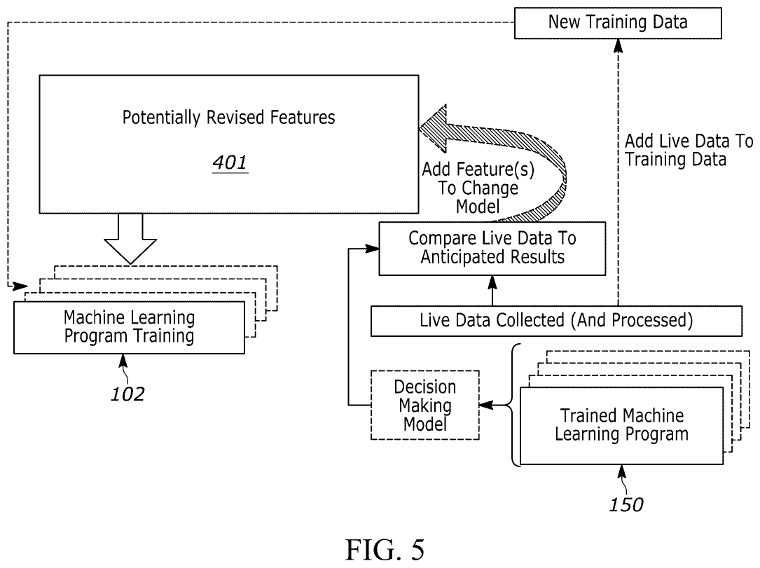

These seed variety objects (seed vectors) 135 play a substantive role in simulation and ML decision making. And, as further illustrated by FIGS. 1A, 1B, 2, 4, and 5, additional data collected may be added to these seed variety objects to create potential revised features (see FIG. 5, 401) toward potentially improving the ability of the models to evaluate and make recommendations with respect to subsequent decisions as to subsequent heterogenous mixtures 110. Seed vectors 135 may include predictive phenotypic or genomic data, such that these predicted phenotypic and/or genomic data may be incorporated into the disclosed simulations and ML decision making. Additionally, data related to genetic marker-based traits may be included in the seed vectors 135 such that information related to genetic marker-based traits may be incorporated into the disclosed simulations and ML decision making.

As illustrated across FIGS. 1A, 1B, 2, 4, and 5, physical testing data may be collected and may be further processed based on directions from the machine learning system 150. As shown in FIGS. 4 and 5, the machine learning model may be updated using live data, such as the directly observed physical data (e.g., genotype, phenotype, agronomic, atmospheric), ingredient processing data, or on one or more derivative data sets based on the observed physical data. These directly observed data may be collected during heterogenous mixture cultivation, field testing, and commercialization and/or from the results of such heterogenous mixture, field testing, and commercialization by, among other things, obtaining tissue samples from the various steps in the process (as illustrated in FIG. 1A).

As should be understood by those skilled in the art based on the foregoing disclosures, there will be a variable number of observations across each of the seed variety objects (or seed vectors) 135 and any related data associated with each specific variety as the system 100 continues to operate. As such, the equal variance assumption that underlies many linear ML models will not be met. Consequently, operations in the present system may better lend themselves to neural network (NN) analysis. NN analysis can include calculations of averages from more than one source. Moreover, because neural network analysis allows the system to capture relationships even where certain outlier values may be “too high” or “too low,” these NNs may provide additional advantages to the system over linear and other models with respect to real-time agricultural decision making.

As illustrated in FIG. 2, the seed record/seed vector 135 may also include the number of actual data points collected with respect to each separate data type, as well as the mean and standard deviation for that data, and various correlations, such as the correlation of observed protein to observed yield. It should be understood by those of ordinary skill in the art having the present disclosure before them that other correlations may be calculated and included in a seed variety object data record 135, such as correlations, if any, between protein and oil, protein and maturity group, protein and food testing data, yield and oil, yield and maturity group, yield and food testing data, oil and maturity group, oil and food testing data, and maturity group and food testing data. It may further be possible using the collected data to identify opportunities to use growing data from one or more prior growing season in predicting future performance of the seed.

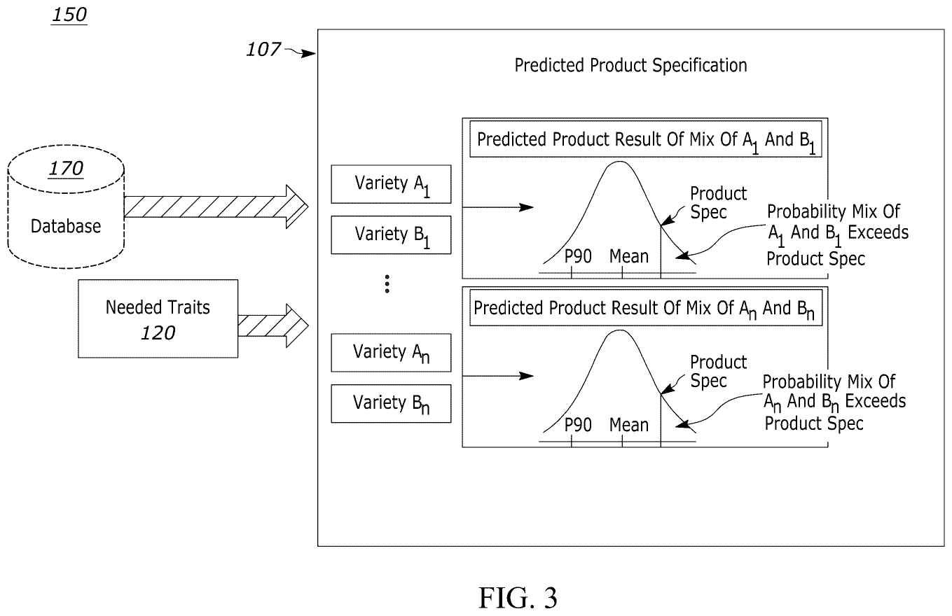

Thus, for example, as shown in FIG. 3, the probabilities with respect to target specifications with specific traits, such as protein and yield can be predicted by the disclosed machine learning model based on data from the database. FIG. 3 depicts the machine learning model 150 using inputs from the database 170 and information related to a target specification 120 to make predictions 107 about which varieties of a plant could be blended in a heterogenous mixture to meet the target specification 120. FIG. 3 generally illustrates that various mixtures of multiple varieties 130 of a plant may be analyzed by the machine learning model 150 to make predictions 107 about an ideal mixture ratio of heterogeneous seed varieties to create, plant (potentially in fields particularly designated by the ML model 150), manage (potentially with particular field management techniques designated by the ML model 150), harvest, and process (potentially in accordance with processing techniques designated by the ML model 150) to meet a given product ingredient specification.

As shown in FIG. 5, it is contemplated that some of the data may be collected and fed back into the model continuously (as potentially revised features (401)). After each data set is collected, it is stored in the database 170 and used to train the machine learning system 150 to continuously improve the machine learning systems' ability to make predictions about preferred heterogenous mixtures 110 of multiple varieties of plants.

The phenotypic data collected by the seed variety objects (or seed vectors) and utilized in the ML system may include various desirable and undesirable traits associated with a particular plant. For example, with respect to plants used in making plant-based protein product phenotypic data may include the protein content in seeds of the plant (measured both in the field using NIR and in a wet lab), the density of other nutrients in the seeds of the plant, the oil content in seeds of the plant, the oleic acid content in seeds of the plant, the fiber content in the seeds of the plant, the oligosaccharides content (e.g., raffinose and stachyose) in seeds of the plant, the saponins/isoflavones/PUFA content in the seeds of the plant, the content of other off-flavor contributing chemicals (e.g., Hexanal and Hexanol) in the seeds of the plant, the moisture content in seeds of the plant (water holding capacity), plant height, the yield history for the plant, the maturity group (MG) of the plant, hilum color, and environmental stress resistance of the plant.

### Computing Environment to Support Machine Learning

As further illustrated by FIG. 1A, data from seed varieties 130, existing specifications 160, desired specification 120, current and historical growing and field conditions (140), processing data 190, and field management techniques 180 are continuous gathered, stored in a database 170, and used to aid the disclosed machine learning system 150 to inform and predict beneficial blended cultivation mixtures to meet product specification requirements 120.

In silico simulations allow the system to test alternatives that cannot be readily tested in the real world because, among other things there are just too many possibilities to test. By picking seed variety objects 135 that are believed to have a desired trait, modeling blended cultivation of more than one seed variety in silico simulations and the various machine learning options, the probability of hitting the desired improved plant-based product increases. The general framework of in silico (stochastic) simulation for plant breeding programs is well-known: See, e.g., Anne Michelle Faux et al., AlphaSim: Software for Breeding Program Simulation, 9(3) THE PLANT GENOME (November 2016), Doi: 10.3835/plantgenome2016.02.0013. PMID: 27902803. (AlphaSim simulates breeding programs in a series of steps: (i) simulate haplotype sequences and pedigree; (ii) drop haplotypes into the base generation of the pedigree and select single-nucleotide polymorphism (SNP) and quantitative trait nucleotide (QTN); (iii) assign QTN effects, calculate genetic values, and simulate phenotypes; (iv) drop haplotypes into the burn-in generations; and (v) perform selection and simulate new generations); Ian Mackay et al., GplusE: beyond genomic selection, 4(1) FOOD ENERGY SEC., 25-35 (Mar. 25, 2015), Doi: 10.1002/fes3.52 (collection of high-throughput phenotype data from field trials (HTP)) (Montes et al. 2007; Araus and Cairns 2014) can be integrated directly into plant breeding programs in a manner analogous to the use of high-density marker data in genomic selection (GS) (Meuwissen et al. 2001; Jannink et al. 2010); Huiming Liu et al., ADAM-Plant: A Software for Stochastic Simulations of Plant Breeding From Molecular to Phenotypic Level and From Simple Selection to Complex Speed Breeding Programs, 9:1926 Front. Plant Sci. (Jan. 9, 2019), Doi: 10.3389/fpls.2018.01926, PMID: 30687343, PMCID, PMC6333911. Each of these articles (among others) describe known systems and software programs that model probable outcomes when crossing and self-pollinating particular plants. These disclosures reference the general possibility of using particular genetic, genotypic and phenotypic data as inputs and provide particular examples for particular plants. These references can generally be extrapolated by one of ordinary skill in the art to other use cases, including soybean. However, none of these references recognize the desirability of assessing the probability that multiple varieties of a given crop, when cultivated together in a heterogenous mixture in a single field can meet specific product specifications faster than through gene editing or selective breeding program alone, especially where that specification has multi-faceted requirements (e.g., higher protein, better taste, improved water holding capacity).

It should also be noted that the machine learning models, data collection, various logic, and/or functions disclosed herein may be enabled using any number of combinations of hardware, firmware, and/or as data and/or instructions embodied in various machine-readable or computer-readable media, in terms of their behavioral, register transfer, logic component, and/or other characteristics. Computer-readable media in which such formatted data and/or instructions may be embodied include, but are not limited to, non-volatile storage media in various forms (e.g., optical, magnetic, or semiconductor storage media) and carrier waves that may be used to transfer such formatted data and/or instructions through wireless, optical, or wired signaling media or any combination thereof. Examples of transfers of such formatted data and/or instructions by carrier waves include, but are not limited to, transfers (uploads, downloads, e-mail, etc.) over the Internet and/or other computer networks via one or more data transfer protocols (e.g., HTTP, FTP, SMTP, and so on).

Aspects of the methods and systems described herein, such as the logic or machine learning models, may be implemented as functionality programmed into any of a variety of circuitry, including programmable logic devices (PLDs), such as field programmable gate arrays (FPGAs), programmable array logic (PAL) devices, electrically programmable logic and memory devices and standard cell-based devices, as well as application specific integrated circuits. Some other possibilities for implementing aspects include: memory devices, microcontrollers with memory (such as EEPROM), embedded microprocessors, firmware, software, etc. Furthermore, aspects may be embodied in microprocessors having software-based circuit emulation, discrete logic (sequential and combinatorial), custom devices, fuzzy (neural) logic, quantum devices, and hybrids of any of the above device types. The underlying device technologies may be provided in a variety of component types, e.g., metal-oxide semiconductor field-effect transistor (MOSFET) technologies like complementary metal-oxide semiconductor (CMOS), bipolar technologies like emitter-coupled logic (ECL), polymer technologies (e.g., silicon-conjugated polymer and metal-conjugated polymer-metal structures), mixed analog and digital, and so on.

Aspects of the methods and systems disclosed herein may be embodied and/or executed by the logic of the processes described herein, which may also be embodied in the form of software instructions and/or firmware that may be executed on any appropriate hardware. For example, logic embodied in the form of software instructions and/or firmware may be executed on a dedicated system or systems, on a personal computer system, on a distributed processing computer system, and/or the like. In some embodiments, logic may be implemented in a stand-alone environment operating on a single computer system and/or logic may be implemented in a networked environment such as a distributed system using multiple computers and/or processors, for example.

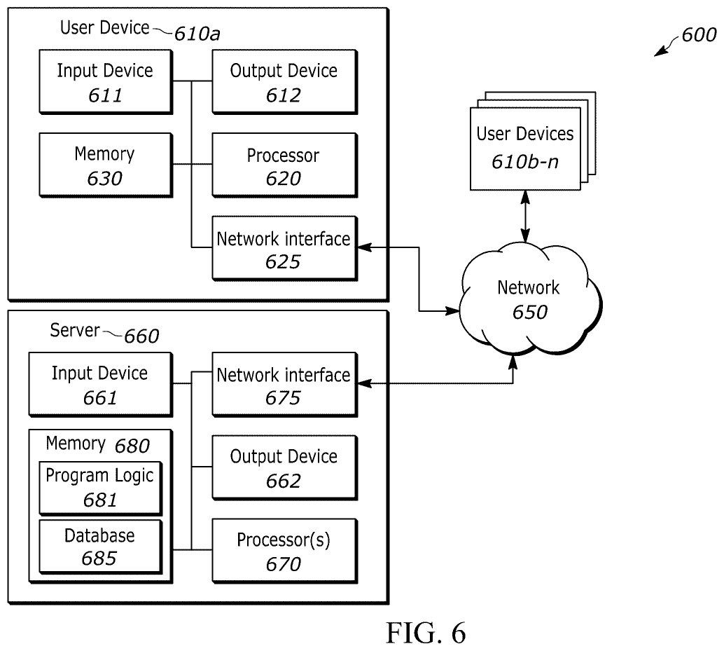

Aspects of the methods and systems described herein may also be implemented on an illustrative system 600, depicted in association with FIG. 6. In particular, system 600 may comprise a user devices 610a-n, server 660, and network 650.

The user device 610 of the system 600 may include various components including, but not limited to, one or more input devices 611, one or more output devices 612, one or more processors 620, a network interface device 625 capable of interfacing with the network 650, one or more non-transitory memories 630 storing processor executable code and/or software application(s), for example including, a web browser capable of accessing a website and/or communicating information and/or data over the network, and/or the like. The memory 630 may also store an application (not shown) that, when executed by the processor 620 causes the user device 610 to provide the functionality of the various systems and methods described the present specification, as would be understood by those of ordinary skill in the art having the present specification, drawings, and claims before them.

The input device 611 may be capable of receiving information input from the user and/or processor 620, and transmitting such information to other components of the user device 610 and/or the network 650. The input device 611 may include, but are not limited to, implementation as a keyboard, touchscreen, mouse, trackball, microphone, remote control, and combinations thereof, for example.

The output device 612 may be capable of outputting information in a form perceivable by the user and/or processor 620. For example, implementations of the output device 612 may include, but are not limited to, a computer monitor, a screen, a touchscreen, an audio speaker, a website, and combinations thereof, for example. It is to be understood that in some exemplary embodiments, the input device 611 and the output device 612 may be implemented as a single device, such as, for example, a computer touchscreen. It is to be further understood that as used herein the term “user” is not limited to a human being, and may comprise, a computer, a server, a website, a processor, a network interface, a user terminal, and combinations thereof, for example.

The server 660 of the system 600 may include various components including, but not limited to, one or more input devices 661, one or more output devices 662, one or more processors 670, a network interface device 675 capable of interfacing with the network 650, and one or more non-transitory memories 680 for storing data structures/tables (including those of database 685) that may be used by the system 600 and particularly server 660 to perform the functions and procedures set forth herein. The memory 680 may also store an application/program store 681 that, when executed by the processor 670, causes the server 660 to provide the functionality of the systems and methods disclosed in the present application.

As shown in FIG. 6, the server 660 may include a single processor or multiple processors working together or independently to execute the program logic 681 stored in the memory 680 as described herein. It is to be understood that, in certain embodiments using more than one processor 670, the processors 670 may be located remotely from one another, located in the same location, or comprising a unitary multi-core processor. The processors 670 may be capable of reading and/or executing processor executable code and/or capable of creating, manipulating, retrieving, altering, and/or storing data structures and data tables (including those of database 685) into the memory 680.

Exemplary embodiments of the processor 670 may be include, but are not limited to, a digital signal processor (DSP), a central processing unit (CPU), a field programmable gate array (FPGA), a microprocessor, a multi-core processor, combinations, thereof, and/or the like, for example. The processor 670 may be capable of communicating with the memory 680 via a path (e.g., data bus). The processor 670 may be capable of communicating with the input device 661 and/or the output device 662.

The input device 661 of the server 660 may be capable of receiving information input from the user and/or processor 670, and transmitting such information to other components of the server 660 and/or the network 650. The input device 661 may include, but are not limited to, implementation as a keyboard, touchscreen, mouse, trackball, microphone, remote control, and/or the like and combinations thereof, for example. The input device 661 may be located in the same physical location as the processor 670, or located remotely and/or partially or completely network-based.

The output device 662 of the server 660 may be capable of outputting information in a form perceivable by the user and/or processor 670. For example, implementations of the output device 662 may include, but are not limited to, a computer monitor, a screen, a touchscreen, an audio speaker, a website, a computer, and/or the like and combinations thereof, for example. The output device 662 may be located with the processor 670, or located remotely and/or partially or completely network-based.

The memory 680 stores applications or program logic 681 as well as data structures (including those of database 685) that may be used by the system 600 and particularly server 660. The memory 680 may be implemented as a conventional non-transitory memory, such as for example, random access memory (RAM), CD-ROM, a hard drive, a solid state drive, a flash drive, a memory card, a DVD-ROM, a disk, an optical drive, combinations thereof, and/or the like, for example. In some embodiments, the memory 680 may be located in the same physical location as the server 660, and/or one or more memory 680 may be located remotely from the server 660. For example, the memory 680 may be located remotely from the server 460 and communicate with the processor 670 via the network 650. Additionally, when more than one memory 680 is used, a first memory 680a may be located in the same physical location as the processor 670, and additional memory 680n may be located in a location physically remote from the processor 670. Additionally, the memory 680 may be implemented as a “cloud” non-transitory computer readable storage memory (i.e., one or more memory 680 may be partially or completely based on or accessed using the network 650).

Each element of the server 660 may be partially or completely network-based or cloud-based, and may or may not be located in a single physical location. As used herein, the terms “network-based,” “cloud-based,” and any variations thereof are intended to include the provision of configurable computational resources on demand via interfacing with a computer and/or computer network, with software and/or data at least partially located on a computer and/or computer network. In other words, the server 660 may or may not be located in single physical location. Additionally, multiple servers 660 may or may not necessarily be located in a single physical location.

Database 685 may comprise one or more data structures and/or data tables stored on non-transitory computer readable storage memory 680 accessible by the processor 670 of the server 660. The database 685 can be a relational database or a non-relational database. Examples of such databases include, but are not limited to, DB2®, Microsoft® Access, Microsoft® SQL Server, Oracle®, mySQL, PostgreSQL, MongoDB, Apache Cassandra, and the like. It should be understood that these examples have been provided for the purposes of illustration only and should not be construed as limiting the presently disclosed inventive concepts. The database 685 can be centralized or distributed across multiple systems.

### Goals and Advantages of Heterogenous Mixtures

The goals of heterogenous mixtures to meet target specifications for food ingredients include, for example, optimizing economics (e.g., with high yield) and skipping processing steps (e.g., low oligosaccharides); empowering ingredient optimization in the field versus at the crush facility may present operational/logistical advantages and enable future farm to feed linkage for specific ingredients; and in-field blending may lead to reduced carbon output vs managing the blend at crush.

In some variations, the varieties are determined and bred to be of similar composition except for protein, oil, and yield. In some variations, the meal or white flake or concentrate or isolate are of similar specifications and performance to the desired homogeneous unblended specification. In some variations, prediction from composite analyses and specifications are used to determine blending ratios. In some variations, in field measures are used to recalculate any shifts to blending in season.

A heterogenous mixture of varieties as provided herein optimize the economics of crop agriculture, especially for producing a harvest product that meets the threshold levels of multiple traits. In certain embodiments, the traits are antagonistically genetically related, for example, protein content and yield and or protein content and oil content. In some embodiments, the threshold levels may be maxima, minima, and exact values.

In some variations, various seed heterogenous mixtures may be used to mitigate the effects of the environmental on the performance of genetics/germplasm.

In some embodiments, the downstream products of the heterogenous mixtures described herein exhibit one or more synergistic complex traits, which may include protein content, yield, oil content, oligosaccharide content, saponins content, amino acid profile, hilum color, and/or a functional property as described herein.

In some embodiments, heterogenous mixtures may yield synergistic effects from mixing varieties with different functionality measurements. Varieties may have a mix of properties, for example, yield performance, water holding capacity (WHC), oil holding capacity (OHC), least gelation concentration (LGC), emulsion capacity (EC), foaming capacity (FC), foaming stability, micro texture, bench texture, or gel texture. In some variations, synergistic effects may be achieved by mixing better functionality varieties with better commercial performance varieties.

In some embodiments, the heterogenous mixtures of varieties described herein yield a blended hilum color which may be desirable to consumers or which may be necessary to meet a particular plant-based ingredient specification. For example, it may be the case that two varieties have difference hilum colors but when these varieties are blended into a heterogenous mixture, the resulting color of the final product will have a color that is a blend of the two initial hilum colors.

In some embodiments, the heterogenous mixtures of varieties described herein yield a net protein pounds per acre advantage as compared to individual varieties. For example, even at a 10 bushels yield drag, the blends of varieties described herein can maintain a significant net protein pounds per acre advantage. Yield refers to bushels (e.g., 60 lbs of grain) per acre; and yield drag refers to the yield achieved relative to comparable market performance. Conventional yield is typically defined as the harvested seed dry matter per unit land area. Post-processing yield is typically the amount of a grain component that is recoverable by processing the grain, e.g., by conventional and/or industrial methods that create ingredients or ingredient intermediates associated with the production of food and/or feed, per unit area of land that was used to grow the beans. For example, the post-processing protein yield produced by a certain variety could be the amount of protein that is recovered in a white flake, a soy protein concentrate, or a soy protein isolate per acre of land that was used to grow the variety. Similarly, the post-harvest oil yield of a variety could be the amount of oil that is recovered from the variety during processing per acre of land used to grow the variety.

In some variations, the post-processing product is derived from processing a final grain heterogenous mixture to produce a food or feed ingredient or product, for example, a protein isolate, other protein product, soybean seed hulls, meal, flake, flour, fiber, sugar, or oil product, or a food, feed, aqua, or industrial product. In some variations, the post-processing property refers to a property of a composition derived from processing a final grain blend to produce a food or feed ingredient or product, for example, a protein isolate, other protein product, soybean seed hulls, meal, flake, flour, fiber, sugar, or oil product, or a food, feed, aqua, or industrial product.

As consumer demand for soy-based food products increases, the need for improved food ingredients has also increased. Current methods to meet these food ingredient specification requirements have relied on gene editing techniques alone or in combination with selective breeding programs. These existing techniques individually and when used in tandem may not achieve the necessary final product specification without significant expenditures of time, money, and other resources. The disclosed system and method 100 present a novel and surprising alternative to achieve new product specifications without relying on these costly methods alone.

By creating a heterogenous mixture (either in a single growing field or across multiple fields) to meet a specific product specification 120, the applicants have achieved something not previously thought possible. Hundreds of years of agricultural practices taught that blended cultivation was impractical, if not impossible. Differences in seed size, plant characteristics, growing requirements, harvesting equipment, transportation, storage, and processing facilities were believed to be variety-specific. The present disclosure 100, with the aid of live, historical, and virtual data, and a data-trained machine learning system 150 now allows for blended cultivation of multiple varieties to meet a specific product specification requirement 120. Moreover, the use of a blended or heterogenous mixture 110 can be implemented throughout the cultivation lifecycle, as depicted in FIG. 8. When, how, and to what degree, blending will occur may be recommended by the machine learning system 150 to meet a particular specification 120.

The disclosed machine learning system 150 and database 170 aid the selection and creation of heterogenous mixtures of multiple varieties of a single plant to meet a specific target product specification 120. As disclosed herein in FIG. 1A, data (135, 145, 165, 185, 195) is collected from multiple sources during the cultivation lifecycle, including data about seeds and varieties 135, fields and field conditions 145, existing specification data 165, field management techniques 185, and from ingredient processing facilities 195. These data are stored in a database 170 and used to iteratively and regularly train 102 the disclosed machine learning system 150. The machine learning system uses the training data and user-selected feature 400 to make predictions 107 about which specific varieties 130 of plant would be ideal candidates for heterogenous mixing. As disclosed herein, in one embodiment, the recommended heterogenous mixture 110 can be a heterogenous mixture of seeds of multiple varieties of a plant species. In other embodiments, the heterogenous mixture can be created during harvesting 196, storage 194, transportation 192, or processing 190 (see FIG. 1A).

The disclosed system and method 100 may be utilized alone or in tandem with other breeding systems, such as gene editing and/or selective breeding programs to meet a target specification 120, as may be recommended by the disclosed machine learning system 150. Alternatively, the model 150 may be able to model and subsequently proscribe combined blending, breeding, gene editing programs that optimize one or more of the probability of meeting/exceeding the product specification, the time to market, the cost of production, environmental impacts, and overall improvement of the germplasm. So, for example, the model 150 may determine that there is a reasonable probability of meeting or exceeding a desired product specification using a proscribed ratio of existing seed varieties Vₐ, V_{b} and V_{c}. The model may further indicate that by breeding V_{b} to V_{b′} and/or V_{c} to V_{c′} and then blending (perhaps even a different ratio) of varieties Vₐ, V_{b′} and V_{c′} that the probability of meeting or exceeding the desired product specification will increase, there will be projected improvement in yield, a reduction in the cost of production (in field management and/or in ingredient processing), and/or a reduction in the environmental impact.

The following non-limiting examples demonstrate additional embodiments of the disclosed system and method.

### Example 1—Theoretical Blend of Two Varieties

In a first example, two varieties of soy, Variety 1 and Variety 2, were prophetically examined as candidates for blended cultivation. These varieties were selected as candidates to blend because they are difficult to crossbreed through existing selective breeding schema. In general, Variety 1 has a higher yield, but relatively lower protein content, whereas Variety 2 has a higher protein content, but corresponding negative yield drag. See FIG. 9A. This made these varieties ideal candidates for heterogenous seed mixture cultivation, because the resulting product is expected to have a relatively high protein content distribution but not suffer as significant of a yield drag overall.

Researchers proposed a 70/30 blend of these varieties (with 70% Variety 1 and 30% Variety 2 by seed weight) to obtain a harvested product that had an increased protein content and a higher relative yield, something that would not be possible through selective breeding of these particular varieties and would have been too time consuming and expensive to attempt through gene editing (even without consideration of the significant risk of failure).

Chart 1, below, shows information related to protein content (of white flake soy product) and yield rates for these two varieties. Chart 2 shows theoretical distributions of protein content for a 70/30 blend of these varieties.

CHART 1
Two Varieties of Soy, Compared

| Variety | Ratio | Mean White Flake Protein % | Yield |
|---|---|---|---|
| Variety 1 | 70% | 60.75 | 51.6 |
| Variety 2 | 30% | 64.4 | 44.75 |

CHART 2
Theoretical Distribution of Protein Content for
a 70/30 Ratio Mixture of Variety 1 and Variety 2

| Distribution | 70/30 Blended White Flake Protein |
|---|---|
| P90 | 59.89 |
| P80 | 60.43 |
| P70 | 61.4 |
| P60 | 61.73 |
| P50 | 62.2 |
| P40 | 62.62 |
| P30 | 63.37 |
| P20 | 64.21 |
| P10 | 64.95 |

A theoretical protein content from 1,000 acres cultivated with a mixture of 70/30 of Variety 1 to Variety 2, and resulted in a white flake content of approximately 61.74. The P50 levels of protein were determined to be 62.2%, higher than the Variety 1 alone expected level (60.75%), and the corresponding yield drag was predicted to be less severe. As will be understood by one of ordinary skill in the art, P90-P10 values depict statistical distribution of a population, where the top 10% of a population has at least the P10 value, the top 20% of the population has the P20 value, the top 30% of the population has the P30 value, and so on. P50 values are used as an average for a population, but the other P values are useful to researchers to understand variation with a population. For example, if a population has a relatively high P10 value and a relatively low P90 value, there will likely be a greater variation in the population. If the P10 and P90 are relatively close to one other, there will be less variation in the population.

### Example 2—Theoretical Blend of Two Varieties of Soy

In another prophetic example, two varieties of soy were mixed. A first variety of soy was an ultra-high protein variety that was low in oligosaccharide. A second variety of soy was high in protein, low in oligosaccharides, and low in saponins, as shown in Chart 3.

CHART 3
Two Varieties of Soy, Compared.

| | Protein | | | | |
|---|---|---|---|---|---|
| Variety | Content | Oligosaccharides | Saponins | | Yield |
| Variety A | 63% | <1% | >5 | mg/kg | −5% Drag |
| Variety B | 61% | <1% | <0.5 | mg/kg | −12% Drag |

Various ratios of the two varieties were examined:

CHART 4
Results of Blended Ratios for Two Varieties of Soy.

| Ratio | Protein | Oligosaccharide | Saponins | | Yield |
|---|---|---|---|---|---|
| 50:50 | 62% | <1% | <2.5 | mg/kg | −8.5% |
| 60:40 | 62.2% | <1% | <3 | mg/kg | −8% |
| 70:30 | 62.5% | <1% | <3.5 | mg/kg | −7% |

As Chart 4 depicts, the result was a product that was high in protein, low in oligosaccharides and low in saponins. The ratios, 50/50, 60/40, and 70/30 that were tested aided researchers to calculate whether a specific food ingredient specification could be obtained from a blended cultivation of these two varieties of soy.

### Example 3—Observed Results From Two Varieties of Soy Blended

FIGS. 9A-D generally relate to Example 2. In Example 2, two varieties of soy mixed according to the present disclosure. In particular, soy varieties Variety 1 and Variety 2 were used in Example 2. Data related to these varieties is known and stored in a database 170, as shown in FIG. 1A. These varieties were chosen because they are difficult to cross-breed because of the protein-yield antagonistic relationship discussed above, as well as other phenotypic differences between the plants as well as other phenotypic differences that make these particular varieties ill-suited for crossbreeding. For example, Variety 1 has a lower average protein content and higher oil content when compared to Variety 2, as shown by FIG. 9A. Furthermore, Variety 1 is approximately 12% larger in seed size when compared to Variety 2, see FIG. 9B. These varieties have also differing flavor profiles, ingredient functionality, and amino acid content (as set forth in the charts below). These differences tend to make it difficult to cross breed these varieties. Conventional planting methods have also avoided co-planting these varieties because of differences in seed size, as well as planting, growing, and processing requirements. However, researchers determined that a heterogenous blend of these varieties could be successfully planted, grown, harvested, and processed in and from the same growing field despite these differences.

Surprisingly, despite the seed size of the Variety 1 being approximately 12% larger than the Variety 2, researchers did not observe the larger Variety 1 seeds migrating to the top or bottom of the heterogeneous seed container 110 (FIG. 1A), during transportation, storage, or planting. Instead, the blend of seeds remained heterogenous throughout even as the seeds were planted. The result was a field with a random distribution of a 60/40 blend of Variety 2 and Variety 1 seeds, respectively. The seeds were planted in Terre Haute, Indiana as part of a field trial program which involved thousands of plants and multiple varieties in April 2023. The plants were managed according to standard practices. In particular, a thoroughly mixed 60/40 blend of the Variety 2 and Variety 1 seeds was created in seed bags using a standard seed counter. Seeds were then transferred to a mechanized planter and planted at a density of 180,000 plants/acre over an area of approximately five acres in standard commercial production settings. Plants were cultivated using standard methods and the resulting grain harvested during the first week of October 2023. Yield data was measured using an on-combine device manufactured by John Deere. Samples of the grain harvested at various points in the field were subjected to NIR analysis to determine protein and oil content on a dry basis.

The heterogenous mix of soybeans were harvested together, stored together, and processed into a white flour product using a single crushing and processing method. The harvested soybeans and defatted flour were analyzed and compared to the predicted results, as shown in the charts below. Moisture content, protein content, oil content, fiber content, ash content for each variety is shown below, as well as predicted content levels for a 60/40 blend as well as observed levels for a 60/40 blend of Variety 2 to Variety 1.

CHART 5
Predicted and Actual Characteristics for Soybeans From a 60/40 Ratio
Mixture of Variety 2 and Variety 1 Soy Plants, Respectively

| | Variety/Sample | | | |
|---|---|---|---|---|
| | Variety | Variety | 60/40 Blend | 60/40 Blend |
| | 2 | 1 | (Theoretical) | (Observed) |
| Moisture (%) | 9.02% | 9.04% | 9.03% | 9.13% |
| Protein (% DB) | 49.31% | 46.63% | 48.24% | 48.35% |
| Crude Fat/Oil (% DB) | 17.98% | 19.07% | 18.42% | 18.27% |
| Crude Fiber (% DB) | 5.70% | 5.40% | 5.58% | 5.70% |
| Ash (% DB) | 5.34% | 5.19% | 5.28% | 5.27% |

As Chart 5 shows, moisture content, protein content, oil content, fiber content, and ash content were analyzed for two varieties of soybeans, Variety 2 and Variety 1, as well as for an actual and a theoretical 60/40 blend of these varieties. The results of a predicted 60/40 blend were predicted and compared to the observed results. The observed 60/40 blend of soybeans was able to achieve 9.13% moisture, 48.38% protein, 18.27% oil, 5.70% fiber, and 5.27% ash content, as compared to the predicted results: 9.03% moisture, 48.24% protein, 18.42% oil, 5.58% fiber, and 5.28% ash content.

CHART 6
Predicted and Actual Characteristics for Defatted Flour From a 60/40
Ratio Mixture of Variety 2 and Variety 1 Soy Plants, Respectively

| | Variety/Sample | | | |
|---|---|---|---|---|
| | Variety | Variety | 60/40 Blend | 60/40 Blend |
| | 2 | 1 | (Theoretical) | (Actual) |
| Moisture (%) | 8.93% | 7.61% | 8.40% | 8.51% |
| Protein (% DB) | 64.58% | 61.29% | 63.26% | 62.78% |
| Crude Fat/Oil (% DB) | 0.63% | 0.63% | 0.63% | 0.54% |
| Crude Fiber (% DB) | 3.10% | 2.70% | 2.94% | 3.10% |
| Ash (% DB) | 6.70% | 6.74% | 6.72% | 6.67% |

Chart 6 shows the moisture content, protein content, oil content, fiber content, and ash content were analyzed for defatted soy flour from two varieties of soy: Variety 2 and Variety 1 as well as for defatted flour from an actual and a theoretical 60/40 blend of these varieties. The results of a predicted 60/40 blend were predicted and compared to the observed results. The observed 60/40 blend of defatted soy flour was able to achieve 8.51% moisture, 62.78% protein, 0.54% oil, 3.10% fiber, and 6.67% ash content, as compared to the predicted results: 8.40% moisture, 63.26% protein, 0.63% oil, 2.94% fiber, and 6.72% ash content.

CHART 7
Predicted and Actual Protein Content and Amino Acid
Composition of Soybeans for a 60/40 Ratio Mixture
of Variety 2 and Variety 1 Soy Plants, Respectively

| | Variety/Sample | | | |
|---|---|---|---|---|
| | Variety | Variety | 60/40 Blend | 60/40 Blend |
| | 2 | 1 | (Theoretical*) | (Actual) |
| Protein (DB) | 49.31% | 46.63% | 48.24% | 48.35% |
| Alanine | 4.08 | 4.18 | 4.12 | 4.14 |
| Arginine | 8.11 | 7.72 | 7.96 | 7.92 |
| Aspartate | 11.48 | 11.43 | 11.46 | 11.44 |
| Cystine/Cysteine | 1.44 | 1.35 | 1.41 | 1.41 |
| Glutamate | 18.80 | 19.06 | 18.90 | 18.99 |
| Glycine | 4.22 | 4.27 | 4.24 | 4.24 |
| Histidine | 2.58 | 2.59 | 2.58 | 2.61 |
| Methionine | 1.46 | 1.44 | 1.45 | 1.45 |
| Proline | 5.05 | 5.15 | 5.09 | 5.21 |
| Serine | 5.09 | 5.00 | 5.05 | 4.96 |
| Tyrosine | 3.24 | 3.30 | 3.27 | 3.31 |
| Valine | 4.66 | 4.87 | 4.74 | 4.78 |
| Threonine | 3.83 | 3.92 | 3.87 | 3.91 |
| Lysine | 6.14 | 6.76 | 6.38 | 6.58 |
| Leucine | 7.50 | 7.76 | 7.60 | 7.63 |
| Isoleucine | 4.38 | 4.59 | 4.46 | 4.55 |
| Phenylalanine | 5.01 | 5.15 | 5.06 | 5.09 |
| Tryptophan | 1.38 | 1.42 | 1.39 | 1.41 |
| Total | 98.46 | 99.96 | 99.03 | 99.61 |

Chart 7 shows results comparing two varieties of soybean amino acid content as well as an observed 60/40 blend of these varieties and a predicted composition for a 60/40 blend of these varieties.

CHART 8
Predicted and Actual Protein Content and Amino Acid Composition
of Defatted Flour for a 60/40 Ratio Mixture of Variety
2 and Variety 1 Soy Plants, respectively

| | Variety/Sample | | | |
|---|---|---|---|---|
| | Variety | Variety | 60/40 Blend | 60/40 Blend |
| | 2 | 1 | (Theoretical) | (Actual) |
| Protein (DB) | 64.58% | 61.29% | 63.26% | 62.78% |
| Alanine | 4.13 | 4.29 | 4.26 | 4.27 |
| Arginine | 8.24 | 7.85 | 8.20 | 8.14 |
| Aspartate | 11.69 | 11.71 | 11.87 | 11.82 |
| Cystine/Cysteine | 1.50 | 1.39 | 1.48 | 1.45 |
| Glutamate | 19.34 | 19.50 | 19.68 | 19.54 |
| Glycine | 4.20 | 4.32 | 4.31 | 4.33 |
| Histidine | 2.62 | 2.63 | 2.66 | 2.64 |
| Methionine | 1.47 | 1.44 | 1.48 | 1.51 |
| Proline | 5.17 | 5.22 | 5.27 | 5.24 |
| Serine | 5.05 | 5.03 | 5.11 | 5.10 |
| Tyrosine | 3.33 | 3.36 | 3.39 | 3.39 |
| Valine | 4.78 | 4.91 | 4.90 | 4.87 |
| Threonine | 3.95 | 3.98 | 4.02 | 3.98 |
| Lysine | 6.55 | 6.44 | 6.60 | 6.39 |
| Leucine | 7.65 | 7.91 | 7.86 | 7.85 |
| Isoleucine | 4.52 | 4.72 | 4.66 | 4.64 |
| Phenylalanine | 5.09 | 5.24 | 5.22 | 5.21 |
| Tryptophan | 1.39 | 1.44 | 1.43 | 1.43 |
| Total | 100.68 | 101.37 | 102.41 | 101.82 |

Chart 8 shows results comparing two varieties of the amino acid content for defatted soybean flour as well as an observed 60/40 blend of these varieties and a predicted composition for a 60/40 blend of these varieties.

CHART 9
Actual Characteristics for Soybeans From a 60/40 Ratio Mixture
of Variety 2 and Variety 1 Soy Plants, Respectively and Predicted
Characteristics for Soybeans from a 70/30 Ratio Mixture of
Variety 2 and Variety 1 Soy Plants, Respectively

| | Variety/Sample | | | |
|---|---|---|---|---|
| | Variety | Variety | 70/30 Blend | 60/40 Blend |
| | 2 | 1 | (Theoretical) | (Actual) |
| Moisture (%) | 9.02% | 9.04% | 9.03% | 9.13% |
| Protein (% DB) | 49.31% | 46.63% | 48.51% | 48.35% |
| Crude Fat/Oil (% DB) | 17.98% | 19.07% | 18.31% | 18.27% |
| Crude Fiber (% DB) | 5.70% | 5.40% | 5.61% | 5.70% |
| Ash (% DB) | 5.34% | 5.19% | 5.30% | 5.27% |

Chart 9 shows moisture content, protein content, oil content, fiber content, and ash content were analyzed for two varieties of soybeans: Variety 2 and Variety 1 as well as for an actual and a theoretical 70/30 blend of these varieties. The results of a predicted 70/30 blend were predicted using the disclosed machine learning system 150 and compared to the observed results. The observed 70/30 blend of soybeans was able to achieve 9.13% moisture, 48.35% protein, 18.27% oil, 5.70% fiber, and 5.27% ash content, as compared to the predicted 9.03% moisture, 48.51% protein, 18.31% oil, 5.61% fiber, and 5.30% ash content.

CHART 10
Actual Characteristics for Defatted Flour from a 60/40 Ratio Mixture
of Variety 2 and Variety 1 Soy Plants, Respectively, and Predicted
Characteristics for Defatted Flour from a 70/30 Ratio Mixture
of Variety 2 and Variety 1 Soy Plants, Respectively

| | Variety/Sample | | | |
|---|---|---|---|---|
| | Variety | Variety | 70/30 Blend | 60/40 Blend |
| | 2 | 1 | (Theoretical) | (Actual) |
| Moisture (%) | 8.93% | 7.61% | 8.53% | 8.51% |
| Protein (% DB) | 64.58% | 61.29% | 63.59% | 63.59% |
| Crude Fat/Oil (% DB) | 0.63% | 0.63% | 0.63% | 0.54% |
| Crude Fiber (% DB) | 3.10% | 2.70% | 2.98% | 3.10% |
| Ash (% DB) | 6.70% | 6.74% | 6.71% | 6.67% |

Chart 10 shows the moisture content, protein content, oil content, fiber content and ash content were analyzed for defatted soy flour from two varieties of soy: Variety 2 and Variety 1 as well as for defatted flour from an actual and a theoretical 70/30 blend of these varieties. The results of a predicted 70/30 blend were predicted using the disclosed machine learning system 150 and compared to the observed results. The observed 70/30 blend of defatted soy flour was able to achieve 8.51% moisture, 63.59% protein, 0.54% oil, 3.10% fiber, and 6.67% ash content, as compared to the predicted results: 8.53% moisture, 63.59% protein, 0.63% oil, 2.98% fiber, and 6.71% ash content.

CHART 11
Actual Protein Content and Amino Acid Composition of Soybeans
from a 60/40 Ratio Mixture of Variety 2 and Variety 1 Soy
Plants, Respectively, and Predicted Protein Content and
Amino Acid Composition of Soybeans from a 60/40 Ratio Mixture
of Variety 2 and Variety 1 Soy Plants, Respectively

| | Variety/Sample | | | |
|---|---|---|---|---|
| | Variety | Variety | 70/30 Blend | 60/40 Blend |
| | 2 | 1 | (Theoretical) | (Actual) |
| Protein (DB) | 49.31% | 46.63% | 48.51% | 48.35% |
| Alanine | 4.08 | 4.18 | 4.11 | 4.14 |
| Arginine | 8.11 | 7.72 | 8.00 | 7.92 |
| Aspartate | 11.48 | 11.43 | 11.46 | 11.44 |
| Cystine/Cysteine | 1.44 | 1.35 | 1.41 | 1.41 |
| Glutamate | 18.80 | 19.06 | 18.87 | 18.99 |
| Glycine | 4.22 | 4.27 | 4.23 | 4.24 |
| Histidine | 2.58 | 2.59 | 2.58 | 2.61 |
| Methionine | 1.46 | 1.44 | 1.45 | 1.45 |
| Proline | 5.05 | 5.15 | 5.08 | 5.21 |
| Serine | 5.09 | 5.00 | 5.06 | 4.96 |
| Tyrosine | 3.24 | 3.30 | 3.26 | 3.31 |
| Valine | 4.66 | 4.87 | 4.72 | 4.78 |
| Threonine | 3.83 | 3.92 | 3.86 | 3.91 |
| Lysine | 6.14 | 6.76 | 6.32 | 6.58 |
| Leucine | 7.50 | 7.76 | 7.58 | 7.63 |
| Isoleucine | 4.38 | 4.59 | 4.44 | 4.55 |
| Phenylalanine | 5.01 | 5.15 | 5.05 | 5.09 |
| Tryptophan | 1.38 | 1.42 | 1.39 | 1.41 |
| Total | 98.46 | 99.96 | 98.88 | 99.61 |

Chart 11 shows results comparing two varieties of the amino acid content for defatted soybean flour as well as an observed 60/40 blend of these varieties and a predicted composition for a 70/30 blend of these varieties.

CHART 12
Actual Protein Content and Amino Acid Composition of Defatted
Flour from a 60/40 Ratio Mixture of Variety 2 and Variety
1 Soy Plants, Respectively, and Predicted Protein Content
and Amino Acid Composition of Defatted Flour from a 60/40
Ratio Mixture of Variety 2 and Variety 1 Soy Plants, Respectively

| | Variety/Sample | | | |
|---|---|---|---|---|
| | Variety | Variety | 70/30 Blend | 60/40 Blend |
| | 2 | 1 | (Theoretical) | (Actual) |
| Protein (DB) | 64.58% | 61.29% | 63.59% | 62.78% |
| Alanine | 4.13 | 4.29 | 4.18 | 4.27 |
| Arginine | 8.24 | 7.85 | 8.13 | 8.14 |
| Aspartate | 11.69 | 11.71 | 11.70 | 11.82 |
| Cystine/Cysteine | 1.50 | 1.39 | 1.47 | 1.45 |
| Glutamate | 19.34 | 19.50 | 19.39 | 19.54 |
| Glycine | 4.20 | 4.32 | 4.23 | 4.33 |
| Histidine | 2.62 | 2.63 | 2.62 | 2.64 |
| Methionine | 1.47 | 1.44 | 1.46 | 1.51 |
| Proline | 5.17 | 5.22 | 5.19 | 5.24 |
| Serine | 5.05 | 5.03 | 5.04 | 5.10 |
| Tyrosine | 3.33 | 3.36 | 3.34 | 3.39 |
| Valine | 4.78 | 4.91 | 4.82 | 4.87 |
| Threonine | 3.95 | 3.98 | 3.96 | 3.98 |
| Lysine | 6.55 | 6.44 | 6.52 | 6.39 |
| Leucine | 7.65 | 7.91 | 7.73 | 7.85 |
| Isoleucine | 4.52 | 4.72 | 4.58 | 4.64 |
| Phenylalanine | 5.09 | 5.24 | 5.14 | 5.21 |
| Tryptophan | 1.39 | 1.44 | 1.41 | 1.43 |
| Total | 100.68 | 101.37 | 100.89 | 101.82 |

Chart 12 shows results comparing two varieties of the amino acid content for defatted soybean flour as well as an observed 60/40 blend of these varieties and a predicted composition for a 70/30 blend of these varieties.

CHART 13
Comparison of White Flour Functionality from Individual Soybean
Varieties Variety 2 and Variety 1 and 60/40 Ratio Mixture
of Variety 2 and Variety 1 Soy Plants, respectively.

| | Variety 2 | Variety 1 | 60/40 Blend |
|---|---|---|---|
| Protein (%) | 59.1 | 57.0 | 58.1 |
| Moisture (%) | 7.0 | 5.4 | 6.1 |
| Water Holding Capacity (g/g Solids) | 1.93 ± 0.02 | 2.14 ± 0.04 | 1.9 ± 0.03 |
| Oil Holding Capacity (g/g Solids) | 1.82 ± 0.03 | 1.82 ± 0.03 | 1.81 ± 0.05 |
| Least Gelation Concentration | 13.0 | 13.0 | 13.0 |
| (% solids) | | | |
| Emulsion Capacity (mL/g Protein) | 1240 ± 28 | 2020 ± 0 | 1680 ± 28 |
| Foaming Capacity (mL/g Protein) | 572 ± 8 | 589 ± 10 | 604 ± 6.9 |
| Foaming Stability (%) | 67.8 ± 0.2 | 68.5 ± 1.9 | 70.4 ± 0.7 |
| Gel Texture (g) (Micro) | 3130.5 ± 201.1 | 3056.8 ± 197.6 | 3075.1 ± 152.2 |
| Gel Texture (g) (Large Scale) | 129 ± 12 | 161 ± 9 | 150 ± 27 |

Chart 13 shows results for white flour from Variety 2 and Variety 1 varieties of soy as well as white flour from a 60/40 blend of these varieties.

CHART 14A
Flavor Profiles of Two Varieties of Soybeans,
Variety 2 and Variety 1, and 60/40 Ratio Mixture
of Variety 2 and Variety 1, Respectively

| | | | | 1-Butanol, |
|---|---|---|---|---|
| | Pentanal | Hexanal | 1-Penten-3-ol | 2/3-methyl |
| Variety 2 | 212.96 | 1646.68 | 391.30 | 11.04 |
| Variety 1 | 317.49 | 3187.60 | 455.86 | 84.46 |
| 60/40 Blend | 263.56 | 1980.02 | 438.48 | 44.24 |

CHART 14B
Flavor Profiles of Two Varieties of Soybeans,
Variety 2 and Variety 1, and 60/40 Ratio Mixture
of Variety 2 and Variety 1, Respectively.

| | 3-Octanone | 1-Hexanol | Nonanal | 2-Octenal |
|---|---|---|---|---|
| Variety 2 | 13.93 | 2257.13 | 24.46 | 20.46 |
| Variety 1 | 12.14 | 2739.56 | 24.37 | 28.88 |
| 60/40 Blend | 13.67 | 2878.34 | 25.91 | 23.56 |

CHART 14C
Flavor Profiles of Two Varieties of Soybeans.
Variety 2 and Variety 1, and 60/40 Ratio Mixture
of Variety 2 and Variety 1, Respectively

| | 2-Hexenal | Furan, 2-Pentyl- | 1-Octen-3-ol | 1-Heptanol |
|---|---|---|---|---|
| Variety 2 | 168.01 | 12.27 | 252.24 | 3.96 |
| Variety 1 | 256.88 | 11.32 | 232.71 | 4.48 |
| 60/40 Blend | 211.78 | 10.22 | 246.46 | 4.39 |

Chart 14A-C shows flavor profile comparison for soy products created from Variety 2 and Variety 1 varieties of soy as well as from a 60/40 blend of these varieties.

CHART 15
Results for Two Varieties of Soybean, Variety 2 and Variety 1, Compared
to Theoretical Average and Lowest Estimate for a 60/40 Ratio Mixture of
Variety 2 and Variety 1, Respectively, Based on Samples from Four Lots

| | Variety/Sample | | | |
|---|---|---|---|---|
| | | | 60/40 Blend - | 60/40 Blend - |
| | Variety 2 | Variety 1 | Theoretical | Theoretical |
| | (Average) | (Average) | (Average) | (Lowest) |
| Moisture (%) | 7.57% | 7.53% | 7.56% | 7.29% |
| | [6.65-9.02] | [6.46-9.04] | | |
| Protein (% Dry) | 49.44% | 46.81% | 48.39% | 47.08% |
| | [48.15-50.61] | [45.48-48.10] | | |
| Crude Fat/Oil | 17.52% | 19.11% | 18.16% | 18.28% |
| (% Dry) | [16.61-17.98] | [18.85-19.82] | | |
| Crude Fiber (% Dry) | 5.05% | 4.85% | 4.97% | 4.22% |
| | [3.90-5.70] | [4.60-5.40] | | |
| Ash (% Dry) | 5.31% | 5.39% | 5.34% | 5.39% |
| | [5.21-5.46] | [5.19-5.71] | | |

Chart 15 shows moisture content, protein content, oil content, fiber content and ash content for two varieties of soy: Variety 2 and Variety 1 as well as theoretical data for a 60/40 blend of these varieties based on an average of four field inputs as well as a theoretical lowest estimated limit.

CHART 16
Results for Two Varieties of Soybean, Variety 2 and Variety 1 Compared
to Theoretical Average and Lowest Estimate for a 60/40 Ratio Mixture of
Variety 2 and Variety 1, Respectively, Based on Samples from Four Lots.

| | | | 60/40 | 60/40 | 60/40 | 60/40 |
|---|---|---|---|---|---|---|
| | | | Blend | Blend | Blend | Blend |
| Variety/ | Variety 2 | Variety 1 | Beans - | WF - | Beans - | WF - |
| Sample | Average | Average | Average | Average | Lowest | Lowest |
| Moisture (%) | 7.57% | 7.53% | 7.56% | N/A | 7.29% | N/A |
| | [6.65-9.02] | [6.46-9.04] | | | | |
| Protein | 49.44% | 46.81% | 48.39% | 63.38% | 47.08% | 61.96% |
| (% Dry) | [48.15-50.61] | [45.48-48.10] | | | | |
| Crude Fat/Oil | 17.52% | 19.11% | 18.16% | N/A | 18.28% | N/A |
| (% Dry) | [16.61-17.98] | [18.85-19.82] | | | | |
| Crude Fiber | 5.05% | 4.85% | 4.97% | N/A | 4.22% | N/A |
| (% Dry) | [3.90-5.70] | [4.60-5.40] | | | | |
| Ash (% Dry) | 5.31% | 5.39% | 5.34% | N/A | 5.39% | N/A |
| | [5.21-5.46] | [5.19-5.71] | | | | |

Chart 16 shows moisture content, protein content, oil content, fiber content and ash content for two varieties of soy: Variety 2 and Variety 1, as well as theoretical data for a 60/40 blend of these varieties based on an average of four field inputs as well as a theoretical lowest estimated limit. Averages for soybeans as well as for resulting white flake are shown.

CHART 17
Amino Acid Composition Comparison Between Two Varieties
of Soy and a Ratio Mixture of Variety 1 and Variety
2, Respectively, Based on Samples from Three Lots.

| | Variety/Sample | | | |
|---|---|---|---|---|
| | | | 40/60 Blend - | 40/60 Blend - |
| | Variety 1 | Variety 2 | Theoretical | Theoretical |
| | (Average) | (Average) | (Average) | (Lowest) |
| Protein (DB) | 46.87% | 49.48% | 48.44% | 47.08% |
| Alanine | 4.10 | 4.11 | 4.11 | 4.21 |
| Arginine | 7.47 | 8.20 | 7.92 | 7.94 |
| Aspartate | 11.46 | 11.59 | 11.54 | 11.95 |
| Cystine/ | 1.26 | 1.30 | 1.28 | 1.33 |
| Cysteine | | | | |
| Glutamate | 18.58 | 18.98 | 18.82 | 19.56 |
| Glycine | 4.22 | 4.22 | 4.22 | 4.32 |
| Histidine | 2.56 | 2.53 | 2.54 | 2.58 |
| Methionine | 1.34 | 1.35 | 1.35 | 1.40 |
| Proline | 4.94 | 4.99 | 4.97 | 5.13 |
| Serine | 5.01 | 5.02 | 5.02 | 5.22 |
| Tyrosine | 3.26 | 3.23 | 3.24 | 3.25 |
| Valine | 4.74 | 4.70 | 4.71 | 4.79 |
| Threonine | 3.78 | 3.82 | 3.81 | 4.01 |
| Lysine | 6.46 | 6.35 | 6.39 | 6.47 |
| Leucine | 7.56 | 7.39 | 7.46 | 7.60 |
| Isoleucine | 4.44 | 4.38 | 4.40 | 4.44 |
| Phenylalanine | 5.01 | 4.90 | 4.95 | 4.98 |
| Tryptophan | 1.42 | 1.38 | 1.40 | 1.40 |
| Total | 97.63 | 98.43 | 98.11 | 100.58 |

Chart 17 shows protein content and amino acid composition for two varieties of soy: Variety 1 and Variety 2 as well as a 40/60 blend of these varieties, respectively. Average predicted amino acid compositions for a 40/60 blend as well as the lowest predicted composition for each amino acid in a 40/60 blend are also shown.

Researchers also compared 40/60 to 60/40 ratios of Variety 1 and Variety 2, as shown in Chart 33, below.

CHART 33
Blended Ratios for 60/40 Ratio Mixture of Variety
2 and Variety 1, Respectively and 40/60 Ratio
Mixture of Variety 2 and Variety 1, Respectively.

| | 60/40 - | 60/40 - | 60/40 - | 40/60 - | 40/60 - | 40/60 - |
|---|---|---|---|---|---|---|
| | Protein | Oil | Yield | Protein | Oil | Yield |
| P90 | 63.5 | 19.82 | 58 | 63.4 | 19.98 | 59.2 |
| P80 | 62.8 | 19.38 | 57.2 | 62.7 | 19.62 | 58.4 |
| P70 | 62.4 | 19.08 | 56 | 62.2 | 19.32 | 57.2 |
| P60 | 62.0 | 18.84 | 55.4 | 61.8 | 19.06 | 56.6 |
| P50 | 61.6 | 18.68 | 55 | 61.2 | 18.92 | 56.2 |
| P40 | 61.1 | 18.44 | 54.6 | 60.7 | 18.66 | 55.8 |
| P30 | 60.4 | 18.18 | 53.8 | 60.0 | 18.42 | 55 |
| P20 | 59.8 | 18 | 52.5 | 59.3 | 18.22 | 53.7 |
| P10 | 58.9 | 17.7 | 50 | 58.3 | 17.9 | 51.2 |

### Example 3—Data Comparing Two Varieties

As shown in the table below, a Commercial Soybean Crusher has validated that processing of a heterogeneous blend can be an effective means to meet a targeted output specification for ingredient manufacturing. Specifically, as shown in the column UHP-Blend it has been validated that this blend works through a crush process.

CHART 18
Actual Data for 60/40, 70/30 Blends and Individual Varieties (V1 and V2) of Soy.

| | 60/40 | 70/30 | | | | | | | |
|---|---|---|---|---|---|---|---|---|---|
| | Blend - | Blend - | V2 WF | | | UHP- | V1 WF | | |
| | WF | WF | Pro | V2 Pro | V2 Oil | Blend | Pro | V1 Pro | V1 Oil |
| P90 | 65.04 | 65.33 | 66.2 | 49.6 | 19.5 | 63.48 | 63.3 | 47 | 20.3 |
| P80 | 64.12 | 64.39 | 65.2 | 49.3 | 18.9 | 62.8 | 62.5 | 46.5 | 20.1 |
| P70 | 63.42 | 63.69 | 64.5 | 48.9 | 18.6 | 62.4 | 61.8 | 46.1 | 19.8 |
| P60 | 62.92 | 63.19 | 64 | 48.7 | 18.4 | 62.02 | 61.3 | 45.9 | 19.5 |
| P50 | 62.24 | 62.53 | 63.4 | 48.3 | 18.2 | 61.58 | 60.5 | 45.4 | 19.4 |
| P40 | 61.56 | 61.82 | 62.6 | 47.9 | 18 | 61.08 | 60 | 45.2 | 19.1 |
| P30 | 60.84 | 61.13 | 62 | 47.5 | 17.7 | 60.42 | 59.1 | 44.7 | 18.9 |
| P20 | 60.14 | 60.43 | 61.3 | 47.1 | 17.5 | 59.78 | 58.4 | 44.3 | 18.7 |
| P10 | 58.98 | 59.31 | 60.3 | 46.5 | 17.3 | 58.92 | 57 | 43.4 | 18.3 |

CHART 19
Comparison of Historical Data for a 60/40 and a 70/30 Ratio
Mixture of Variety 2 and Variety 1 of Soy, respectively.

| | P90 | P80 | P70 | P60 | P50 | P40 | P30 | P20 | P10 |
|---|---|---|---|---|---|---|---|---|---|
| 60/40 - % Protein | 59.94 | 60.80 | 61.42 | 61.84 | 62.14 | 62.66 | 63.20 | 63.72 | 65.60 |
| 70/30 - % Protein | 60.28 | 61.05 | 61.69 | 62.13 | 62.43 | 62.97 | 63.50 | 63.99 | 66.13 |

Chart 19 shows actual protein content for a 60/40 and 70/30 blend of Variety 2 and Variety 1, respectively.

### Example 4—Exemplary Blend Calculations

Example 4 demonstrates various blends of multiple varies at several ratios. In particular, blended combinations to show mixtures of Variety 3 vs. Variety 4 (“Blend 1”), Variety 5 vs. Variety 6 (“Blend 2”), Variety 7 vs. Variety 4 (“Blend 3”) and Variety 3 vs. Variety 7 (“Blend 4”), and are shown in the charts below. Researchers amassed data regarding soybean varieties as disclosed herein and used that data to generate the following charts.

Blend 1 used data from two proprietary varieties of soy: Variety 3 and Variety 4 to generate the following data related to protein content, oil content and yield.

CHART 20
Blend 1 - Variety 3 vs. Variety 4

| | P90 | P80 | P70 | P60 | P50 | P40 | P30 | P20 | P10 | |
|---|---|---|---|---|---|---|---|---|---|---|
| Variety 3 | 41.62 | 40.73 | 40.02 | 39.29 | 39.14 | 38.92 | 38.49 | 38.08 | 37.71 | Protein |
| Variety 4 | 47.98 | 47.38 | 47.02 | 46.48 | 46.18 | 46.04 | 45.56 | 44.98 | 44.65 | Protein |
| Variety 3 | 22.67 | 22.43 | 22.16 | 21.7 | 21.63 | 21.56 | 21.46 | 21.23 | 20.72 | Oil |
| Variety 4 | 19.3 | 18.85 | 18.77 | 18.7 | 18.47 | 18.42 | 18.08 | 18.03 | 17.78 | Oil |
| Variety 3 | 79.1 | 77.4 | 74.7 | 73.4 | 71.7 | 68.7 | 62.4 | 58.75 | 50.3 | Yield |
| Variety 4 | 79.65 | 70.97 | 69.82 | 65.85 | 62 | 61.17 | 58.7 | 58.5 | 49.8 | Yield |

Data from Chart 16 were used to make predictions regarding an 80/20, 70/30, and 60/40 ratio of these varieties. These data were used to make prediction about blended composition for these varieties.

CHART 21A
Blend 1 Calculation 1 - 80/20 Ratio of Variety 3 vs. Variety 4

| Ratio | Protein | Oil | Yield | WF Protein |
|---|---|---|---|---|
| P90 | 42.9 | 22.0 | 79.2 | |
| P80 | 42.1 | 21.7 | 76.1 | |
| P70 | 41.4 | 21.5 | 73.7 | |
| P60 | 40.7 | 21.1 | 71.9 | |
| P50 | 40.5 | 21.0 | 69.8 | 54.30% |
| P40 | 40.3 | 20.9 | 67.2 | |
| P30 | 39.9 | 20.8 | 61.7 | |
| P20 | 39.5 | 20.8 | 58.7 | |
| P10 | 39.1 | 20.1 | 50.2 | |

CHART 21B
Predicted Blend Results - 80/20 Ratio of Variety 3 vs. Variety 4

| | | | White Flake | |
|---|---|---|---|---|
| Protein % | Fat % | Hulls % | Fat % | White Flake Protein |
| (Dry) | (Dry) | (Estimate) | (Estimate) | (Dry) (Estimate) |
| 38.7% | 22.7% | 6.5% | 0.60% | 54.30% |

CHART 22A
Blend 1 Calculation 2 - 70/30 Ratio of Variety 3 vs. Variety 4

| Ratio | Protein | Oil | Yield | WF Protein |
|---|---|---|---|---|
| P90 | 42.9 | 22.0 | 79.2 | |
| P80 | 42.1 | 21.7 | 76.1 | |
| P70 | 41.4 | 21.5 | 73.7 | |
| P60 | 40.7 | 21.1 | 71.9 | |
| P50 | 40.5 | 21.0 | 69.8 | 54.30% |
| P40 | 40.3 | 20.9 | 67.2 | |
| P30 | 39.9 | 20.8 | 61.7 | |
| P20 | 39.5 | 20.8 | 58.7 | |
| P10 | 39.1 | 20.1 | 50.2 | |

CHART 22B
Predicted Blend Results - 70/30 Ratio of Variety 3 vs. Variety 4

| | | | White Flake | |
|---|---|---|---|---|
| Protein % | Fat % | Hulls % | Fat % | White Flake Protein |
| (Dry) | (Dry) | (Estimate) | (Estimate) | (Dry) (Estimate) |
| 41.3% | 20.7% | 6.5% | 0.60% | 56.28% |

CHART 23A
Blend 1 Calculation 3- 60/40 Ratio of Variety 3 vs. Variety 4

| Ratio | Protein | Oil | Yield | WF Protein |
|---|---|---|---|---|
| P90 | 44.2 | 21.3 | 79.3 | |
| P80 | 43.4 | 21.0 | 74.8 | |
| P70 | 42.8 | 20.8 | 72.7 | |
| P60 | 42.2 | 20.5 | 70.4 | |
| P50 | 42.0 | 20.4 | 67.8 | 56.99% |
| P40 | 41.8 | 20.3 | 65.7 | |
| P30 | 41.3 | 20.1 | 60.9 | |
| P20 | 40.8 | 20.1 | 58.7 | |
| P10 | 40.5 | 19.5 | 50.1 | |

CHART 23B
Predicted Blend Results - 60/40 Ratio of Variety 3 vs. Variety 4

| | | | White Flake | |
|---|---|---|---|---|
| Protein % | Fat % | Hulls % | Fat % | White Flake Protein |
| (Dry) | (Dry) | (Estimate) | (Estimate) | (Dry) (Estimate) |
| 42.0% | 20.4% | 6.5% | 0.60% | 56.99% |

This process was repeated for additional combinations of soy varieties. A second blend (“Blend 2”) is shown below for Variety 5 and Variety 6.

CHART 24
Blend 2 - Variety 5 vs. Variety 6.

| | P90 | P80 | P70 | P60 | P50 | P40 | P30 | P20 | P10 | |
|---|---|---|---|---|---|---|---|---|---|---|
| Variety 5 | 45.73 | 45.02 | 44.76 | 44.44 | 44.15 | 43.88 | 43.48 | 43.31 | 42.76 | Protein |
| Variety 6 | 40.87 | 40.08 | 39.62 | 39.42 | 38.93 | 38.57 | 38 | 37.79 | 36.6 | Protein |
| Variety 5 | 19.95 | 19.66 | 19.53 | 19.4 | 19.15 | 19.03 | 18.91 | 18.86 | 18.34 | Oil |
| Variety 6 | 22.9 | 22.55 | 22.46 | 22.24 | 22.05 | 21.82 | 21.75 | 21.66 | 21.37 | Oil |
| Variety 5 | 70.97 | 68.43 | 66.07 | 63.35 | 60.8 | 57.59 | 55.12 | 52.82 | 47.33 | Yield |
| Variety 6 | 80.9 | 76.6 | 73.45 | 73.1 | 68 | 65.85 | 60.1 | 55.55 | 52.65 | Yield |

Data from Chart 20 were used to make predictions regarding a 70/30, 60/40, and 50/50 ratio of these varieties:

CHART 25A
Blend 2 Calculation 1 - 70/30 Ratio of Variety 5 vs. Variety 6

| Ratio | Protein | Oil | Yield | WF Protein |
|---|---|---|---|---|
| P90 | 44.3 | 20.8 | 73.9 | |
| P80 | 43.5 | 20.5 | 70.9 | |
| P70 | 43.2 | 20.4 | 68.3 | |
| P60 | 42.9 | 20.3 | 66.3 | |
| P50 | 42.6 | 20.0 | 63.0 | 57.57% |
| P40 | 42.3 | 19.9 | 60.1 | |
| P30 | 41.8 | 19.8 | 56.6 | |
| P20 | 41.7 | 19.8 | 53.6 | |
| P10 | 40.9 | 19.2 | 48.9 | |

CHART 25B
Predicted Blend Results - 70/30 Ratio of Variety 5 vs. Variety 6

| | | | White Flake | |
|---|---|---|---|---|
| Protein % | Fat % | Hulls % | Fat % | White Flake Protein |
| (Dry) | (Dry) | (Estimate) | (Estimate) | (Dry) (Estimate) |
| 42.6% | 20.0% | 6.5% | 0.60% | 57.57% |

CHART 26
Blend 2 Calculation 2 - 60/40 Ratio of Variety 5 vs. Variety 6

| Ratio | Protein | Oil | Yield | WF Protein |
|---|---|---|---|---|
| P90 | 43.8 | 21.1 | 74.9 | |
| P80 | 43.0 | 20.8 | 71.7 | |
| P70 | 42.7 | 20.7 | 69.0 | |
| P60 | 42.4 | 20.5 | 67.3 | |
| P50 | 42.1 | 20.3 | 63.7 | 57.09% |
| P40 | 41.8 | 20.1 | 60.9 | |
| P30 | 41.3 | 20.0 | 57.1 | |
| P20 | 41.1 | 20.0 | 53.9 | |
| P10 | 40.3 | 19.6 | 49.5 | |

CHART 26B
Predicted Blend Results - 60/40 Ratio of Variety 5 vs. Variety 6

| | | | White Flake | White Flake |
|---|---|---|---|---|
| Protein % | Fat % | Hulls % | Fat % | Protein (Dry) |
| (Dry) | (Dry) | (Estimate) | (Estimate) | (Estimate) |
| 42.1% | 20.3% | 6.5% | 0.60% | 57.09% |

CHART 27A
Blend 2 Calculator 3- 50/50 Ratio of Variety 5 vs. Variety 6

| Ratio | Protein | Oil | Yield | WF Protein |
|---|---|---|---|---|
| P90 | 43.3 | 21.4 | 75.9 | |
| P80 | 42.6 | 21.1 | 72.5 | |
| P70 | 42.2 | 21.0 | 69.8 | |
| P60 | 41.9 | 20.8 | 68.2 | |
| P50 | 41.5 | 20.6 | 64.4 | 56.61% |
| P40 | 41.2 | 20.4 | 61.7 | |
| P30 | 40.7 | 20.3 | 57.6 | |
| P20 | 40.6 | 20.3 | 54.2 | |
| P10 | 39.7 | 19.9 | 50.0 | |

CHART 27B
Predicted Blend Results - 50/50 Ratio of Variety 5 vs. Variety 6

| | | | White Flake | White Flake |
|---|---|---|---|---|
| Protein % | Fat % | Hulls % | Fat % | Protein (Dry) |
| (Dry) | (Dry) | (Estimate) | (Estimate) | (Estimate) |
| 41.5% | 20.6% | 6.5% | 0.60% | 56.61% |

This process was repeated for additional combinations of soy varieties. A third blend (“Blend 3”) is shown below for Variety 7 and Variety 4.

CHART 28
Blend 3 -Variety 7 vs. Variety 4

| | P90 | P80 | P70 | P60 | P50 | P40 | P30 | P20 | P10 | |
|---|---|---|---|---|---|---|---|---|---|---|
| Variety 7 | 45.8 | 45.04 | 44.78 | 44.56 | 44.51 | 44.42 | 44.11 | 43.89 | 42.91 | Protein |
| Variety 4 | 47.98 | 47.38 | 47.02 | 46.48 | 46.18 | 46.04 | 45.56 | 44.98 | 44.65 | Protein |
| Variety 7 | 19.89 | 19.58 | 19.48 | 19.43 | 19.17 | 19.08 | 18.91 | 18.72 | 18.36 | Oil |
| Variety 4 | 19.3 | 18.85 | 18.77 | 18.7 | 18.47 | 18.42 | 18.08 | 18.03 | 17.78 | Oil |
| Variety 7 | 75.3 | 72 | 67.7 | 65 | 63.3 | 59.23 | 56.55 | 53.85 | 50 | Yield |
| Variety 4 | 79.65 | 70.97 | 69.82 | 65.85 | 62 | 61.17 | 58.7 | 58.5 | 49.8 | Yield |

Data from Chart 25 were used to make predictions regarding a 70/30, 60/40, and 50/50 ratio of these varieties:

CHART 29A
Blend 3 Calculation 1 - 70/30 Ratio of Variety 7 vs. Variety 4

| Ratio | Protein | Oil | Yield | WF Protein |
|---|---|---|---|---|
| P90 | 46.5 | 19.7 | 76.6 | |
| P80 | 45.7 | 19.4 | 71.7 | |
| P70 | 45.5 | 19.3 | 68.3 | |
| P60 | 45.1 | 19.2 | 65.3 | |
| P50 | 45.0 | 19.0 | 62.9 | 59.99% |
| P40 | 44.9 | 18.9 | 59.8 | |
| P30 | 44.5 | 18.7 | 57.2 | |
| P20 | 44.2 | 18.7 | 55.2 | |
| P10 | 43.4 | 18.2 | 49.9 | |

CHART 29B
Predicted Blend Results - 70/30 Ratio of Variety 7 vs. Variety 4

| | | | White Flake | White Flake |
|---|---|---|---|---|
| Protein % | Fat % | Hulls % | Fat % | Protein (Dry) |
| (Dry) | (Dry) | (Estimate) | (Estimate) | (Estimate) |
| 45.0% | 19.0% | 6.5% | 0.60% | 59.99% |

CHART 30A
Blend 3 Calculation 2 - 60/40 Ratio of Variety 7 vs. Variety 4

| Ratio | Protein | Oil | Yield | WF Protein |
|---|---|---|---|---|
| P90 | 46.7 | 19.7 | 77.0 | |
| P80 | 46.0 | 19.3 | 71.6 | |
| P70 | 45.7 | 19.2 | 68.5 | |
| P60 | 45.3 | 19.1 | 65.3 | |
| P50 | 45.2 | 18.9 | 62.8 | 60.16% |
| P40 | 45.1 | 18.8 | 60.0 | |
| P30 | 44.7 | 18.6 | 57.4 | |
| P20 | 44.3 | 18.6 | 55.7 | |
| P10 | 43.6 | 18.1 | 49.9 | |

CHART 30B
Predicted Blend Results - 60/40 Ratio of Variety 7 vs. Variety 4

| | | | White Flake | White Flake |
|---|---|---|---|---|
| Protein % | Fat % | Hulls % | Fat % | Protein (Dry) |
| (Dry) | (Dry) | (Estimate) | (Estimate) | (Estimate) |
| 45.2% | 18.9% | 6.5% | 0.60% | 60.16% |

CHART 31A
Blend 3 Calculation 3- 50/50 Ratio of Variety 7 vs. Variety 4

| Ratio | Protein | Oil | Yield | WF Protein |
|---|---|---|---|---|
| P90 | 46.9 | 19.6 | 77.5 | |
| P80 | 46.2 | 19.2 | 71.5 | |
| P70 | 45.9 | 19.1 | 68.8 | |
| P60 | 45.5 | 19.1 | 65.4 | |
| P50 | 45.3 | 18.8 | 62.7 | 60.32% |
| P40 | 45.2 | 18.8 | 60.2 | |
| P30 | 44.8 | 18.5 | 57.6 | |
| P20 | 44.4 | 18.5 | 56.2 | |
| P10 | 43.8 | 18.1 | 49.9 | |

CHART 31B
Predicted Blend Results - 50/50 Ratio of Variety 7 vs. Variety 4

| | | | White Flake | White Flake |
|---|---|---|---|---|
| Protein % | Fat % | Hulls % | Fat % | Protein (Dry) |
| (Dry) | (Dry) | (Estimate) | (Estimate) | (Estimate) |
| 45.3% | 18.8% | 6.5% | 0.60% | 60.32% |

This process was repeated for additional combinations of soy varieties. A fourth blend (“Blend 4”) is shown below for Variety 3 and Variety 7.

CHART 32
Blend 4 - Variety 3 vs. Variety 7

| | P90 | P80 | P70 | P60 | P50 | P40 | P30 | P20 | P10 | |
|---|---|---|---|---|---|---|---|---|---|---|
| Variety 3 | 41.62 | 40.73 | 40.02 | 39.29 | 39.14 | 38.92 | 38.49 | 38.08 | 37.71 | Protein |
| Variety 7 | 45.8 | 45.04 | 44.78 | 44.56 | 44.51 | 44.42 | 44.11 | 43.89 | 42.91 | Protein |
| Variety 3 | 22.67 | 22.43 | 22.16 | 21.7 | 21.63 | 21.56 | 21.46 | 21.23 | 20.72 | Oil |
| Variety 7 | 19.89 | 19.58 | 19.48 | 19.43 | 19.17 | 19.08 | 18.91 | 18.72 | 18.36 | Oil |
| Variety 3 | 79.1 | 77.4 | 74.7 | 73.4 | 71.7 | 68.7 | 62.4 | 58.75 | 50.3 | Yield |
| Variety 7 | 75.3 | 72 | 67.7 | 65 | 63.3 | 59.23 | 56.55 | 53.85 | 50 | Yield |

Data from Chart 29 were used to make predictions regarding an 80/20, 70/30, and 60/40 ratio of these varieties:

CHART 33A
Blend 4 Calculation 1 - 80/20 Ratio of Variety 3 vs. Variety 7

| Ratio | Protein | Oil | Yield | WF Protein |
|---|---|---|---|---|
| P90 | 42.5 | 22.1 | 78.3 | |
| P80 | 41.6 | 21.9 | 76.3 | |
| P70 | 41.0 | 21.6 | 73.3 | |
| P60 | 40.3 | 21.2 | 71.7 | |
| P50 | 40.2 | 21.1 | 70.0 | 55.21% |
| P40 | 40.0 | 21.1 | 66.8 | |
| P30 | 39.6 | 21.0 | 61.2 | |
| P20 | 39.2 | 21.0 | 57.8 | |
| P10 | 38.8 | 20.2 | 50.2 | |

CHART 33B
Predicted Blend Results - 80/20 Ratio of Variety 3 vs. Variety 7

| | | | White Flake | White Flake |
|---|---|---|---|---|
| Protein % | Fat % | Hulls % | Fat % | Protein (Dry) |
| (Dry) | (Dry) | (Estimate) | (Estimate) | (Estimate) |
| 40.2% | 21.1% | 6.5% | 0.60% | 55.21% |

CHART 34A
Blend 4 Calculation 2 - 70/30 Ratio of Variety 3 vs. Variety 7

| Ratio | Protein | Oil | Yield | WF Protein |
|---|---|---|---|---|
| P90 | 42.9 | 21.8 | 78.0 | |
| P80 | 42.0 | 21.6 | 75.8 | |
| P70 | 41.4 | 21.4 | 72.6 | |
| P60 | 40.9 | 21.0 | 70.9 | |
| P50 | 40.8 | 20.9 | 69.2 | 55.76% |
| P40 | 40.6 | 20.8 | 65.9 | |
| P30 | 40.2 | 20.7 | 60.6 | |
| P20 | 39.8 | 20.7 | 57.3 | |
| P10 | 39.3 | 20.0 | 50.2 | |

CHART 34B
Predicted Blend Results - 70/30 Ratio of Variety 3 vs. Variety 7

| | | | White Flake | White Flake |
|---|---|---|---|---|
| Protein % | Fat % | Hulls % | Fat % | Protein (Dry) |
| (Dry) | (Dry) | (Estimate) | (Estimate) | (Estimate) |
| 40.8% | 20.9% | 6.5% | 0.60% | 55.76% |

CHART 35A
Blend 4 Calculator 3 - 60/40 Variety 3 vs. Variety 7

| Ratio | Protein | Oil | Yield | WF Protein |
|---|---|---|---|---|
| P90 | 43.3 | 21.6 | 77.6 | |
| P80 | 42.5 | 21.3 | 75.2 | |
| P70 | 41.9 | 21.1 | 71.9 | |
| P60 | 41.4 | 20.8 | 70.0 | |
| P50 | 41.3 | 20.6 | 68.3 | 56.30% |
| P40 | 41.1 | 20.6 | 64.9 | |
| P30 | 40.7 | 20.4 | 60.1 | |
| P20 | 40.4 | 20.4 | 56.8 | |
| P10 | 39.8 | 19.8 | 50.2 | |

CHART 35B
Predicted Blend Results - 60/40 Ratio of Variety 3 vs. Variety 7

| | | | White Flake | White Flake |
|---|---|---|---|---|
| Protein % | Fat % | Hulls % | Fat % | Protein (Dry) |
| (Dry) | (Dry) | (Estimate) | (Estimate) | (Estimate) |
| 40.8% | 20.9% | 6.5% | 0.60% | 55.76% |

These results can be compared against a food ingredient specification, and the disclosed machine learning system can predict which combination of which variety and in which ratio would be best suited to meet that particular specification.

The teachings herein are not limited to certain plant species, and it is envisioned that they can be modified to be useful for monocots, dicots, and/or substantially any crop and/or valuable plant type, including plants that can reproduce by self-fertilization and/or cross fertilization, hybrids, inbreds, varieties, and/or cultivars thereof. Some of example plant species include, soybeans (Glycine max), peas (Pisum sativum and other members of the Fabaceae like Cjanus and Vigna species), chickpeas (Cicer arietinum), peanuts (Arachis hypogaea), lentils (Lens culinaris or Lens esculenta), lupins (various Lupinus species), mesquite (various Proopis species), clover (various Trifolium species), carob (Ceratonia siliqua), tamarind, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), camelina (Camelina sativa), millet (e.g., pearl millet (Pennisetum glaucum), proso millet (Panicum miliaceum), foxtail millet (Setaria italica), finger millet (Eleusine coracana)), sunflower (Helianthus annuus), quinoa (Chenopodium quinoa), chicory (Cichorium intybus), tomato (Solanum lycopersicum), lettuce (Lactuca sativa), safflower (Carthamus tinctorius), wheat (Triticum aestivum), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oil palm (Elaeis guineensis), poplar (Populus spp.), eucalyptus (Eucalyptus spp.), oats (Avena sativa), barley (Hordeum vulgare), flax (Linum usitatissimum), Buckwheat (Fagopyrum esculentum) vegetables, ornamentals, and conifers.

While particular embodiments of the present invention have been shown and described, it should be noted that changes and modifications may be made without departing from the presently disclosed inventive concepts in its broader aspects and, therefore, the aim in the appended claims is to cover all such changes and modifications as fall within the true spirit and scope of this invention.

1. A method for training a machine-learning model and subsequently applying that machine learning model to produce a plant-based product, the plant-based product having a functional specification with a first threshold value of a first trait and a second threshold value of a second trait, the method comprising:
collecting training data into a database, with a processor, wherein such training data comprises seed data for a plurality of seed varieties, such seed data including a first phenotypic trait and a second phenotypic trait;
training, with the processor, a first neural network machine learning model using the training data in the database based on a plurality of selected features including the first and second phenotypic traits;
inputting, via the processor, the first and second threshold values into the trained first neural network machine learning model; and
generating a list of heterogeneous mixture ratios of at least a first seed variety and a second seed variety ranked based on an aggregate probability that the heterogeneous mixture will achieve the functional specification for the plant-based product after processing of the first and second seed varieties in the heterogeneous mixture ratio.

2. The method of claim 1 further comprising mixing the first seed variety and the second seed variety into a first heterogeneous mixture based on a first ratio selected from the list of heterogeneous mixture ratios.

3. The method of claim 2 further comprising processing the first heterogeneous mixture into the plant-based product.

4. The method of claim 2 further comprising:
planting the first heterogeneous mixture of seeds in a first agricultural field;
managing plants grown from the first heterogeneous mixture seeds in the first agricultural field, wherein the plants produce either the first seed variety or the second seed variety;
harvesting the first and second seed varieties from the plants in the first agricultural field; and
processing the first and second seed varieties harvest from the first agricultural field into the plant-based product.

5. The method of claim 4 wherein such training data further comprises field conditions for a plurality of agricultural fields, such field conditions including a first field condition and the plurality of selected features including the first field condition, the method further comprising designating the first agricultural field for planting the first heterogeneous mixture of seeds whereby the aggregate probability that the first heterogeneous mixture will achieve the functional specification of the plant-based product after processing of the first and second seed varieties from the plants in the first agricultural field takes into account the field conditions of the first agricultural field.

6. The method of claim 5 wherein such training data further comprises field management techniques, such field management techniques including a first field management technique and the plurality of selected features including the first field management technique, further comprising providing instructions for use the first field management technique in association with the first heterogeneous mixture of seeds in the first agricultural field whereby the aggregate probability that the first heterogeneous mixture will achieve the functional specification of the plant-based product after processing of the first and second seed varieties from the plants in the first agricultural field takes into account the first field management technique.

7. The method of claim 3 further comprising collecting data, by the processor, from the plant-based product after processing of the first and second seed varieties and such training data further comprises produced product specification data.

8. The method of claim 2 further comprising breeding for the second seed variety by crossing a third seed variety and a fourth seed variety as instructed by the first neural network machine learning model.

9. The method of claim 1, wherein the first phenotypic data is predicted data.

10. The method of claim 1, wherein the first phenotypic data is genetic characterization data.

11. The method of claim 1, wherein the first trait is a genetic marker-based trait.
